(19) United States
(12) Patent Application Publication
(10) Pub. No.: US 2025/0041425 A1
(43) Pub. Date: Feb. 6, 2025

(54) EXCIPIENT GRANULATIONS

(71) Applicant: XWPHARMA LTD., REDWOOD CITY, CA (US)

(72) Inventors: SAMI KARABORNI, CUPERTINO, CA (US); DANIEL M. CANAFAX, HALF MOON BAY, CA (US); JIA-NING XIANG, FREMONT, CA (US); WILLIAM W. XIANG, FREMONT, CA (US)

(21) Appl. No.: 18/789,859

(22) Filed: Jul. 31, 2024

Related U.S. Application Data
(60) Provisional application No. 63/529,880, filed on Jul. 31, 2023.

(51) Int. Cl. A61K 47/36; A61K 9/00; A61K 9/08; A61K 45/06; A61K 47/34

(52) U.S. Cl. CPC A61K 47/36; A61K 9/0053; A61K 9/08; A61K 45/06; A61K 47/34

(57) ABSTRACT

Excipient granulations containing a viscosifying agent, a disintegrant, and one or more additional excipients are disclosed. An excipient granulation can be combined with a pharmaceutical granulation to provide a pharmaceutical composition. The excipient granulations can be used to increase the viscosity of an aqueous composition such as an oral pharmaceutical composition. When added to an aqueous solution, the excipient granulation can dissolve to provide a suspension of the pharmaceutical granules in a viscous solution. The excipient granulation can be used to improve the palatability of oral pharmaceutical compositions containing a pharmaceutical granulation.

This application claims the benefit under 35 U.S.C. § 119(e) of U.S. Provisional Application No. 63/529,880 filed on Jul. 31, 2023, which is incorporated by reference in its entirety.

### FIELD

The disclosure relates to excipient granulations, compositions comprising the excipient granulations, and the use of the excipient granulations. The excipient granulations can be used to improve the palatability of an oral pharmaceutical composition.

### BACKGROUND

Active pharmaceutical ingredients can be administered as pharmaceutical granulations. To improve the palatability of pharmaceutical granulations administered to a patient a pharmaceutical granulation can be dispersed and/or suspended in a viscous solution. It can be convenient to provide viscosifying agents and additional excipients along with the pharmaceutical granulation such that the viscosifying agent, additional excipients, and the pharmaceutical granulation can be combined with water to provide a palatable oral pharmaceutical composition.

### SUMMARY

According to the present invention, an excipient granulation comprises a plurality of excipient granules, wherein the excipient granules comprise: a viscosifying agent; a disintegrant; and one or more additional excipients.

According to the present invention, method of making the excipient granules comprises: dry mixing the viscosifying agent, the disintegrant, and the one or more excipients to provide a dry mixture; granulating the dry mixture with a buffer solution to provide a first granulation; wet massing the first granulation to form a first wet mass; granulating the first wet mass with water to provide a second granulation; wet massing the second granulation to form a second wet mass; and drying the second wet mass to provide the excipient granules.

According to the present invention, an excipient granulation comprises excipient granules prepared according to the present invention.

According to the present invention, a composition comprises an excipient granulation according to the present invention.

According to the present invention, a pharmaceutical composition comprises: an excipient granulation according to the present invention; and an active pharmaceutical ingredient.

According to the present invention, a product comprises an excipient granulation according to the present invention or a composition according to the present invention.

According to the present invention, a pharmaceutical product comprises a pharmaceutical composition according to the present invention.

According to the present invention, a composition for oral administration is prepared using an excipient granulation according to the present invention.

According to the present invention, an oral pharmaceutical composition for oral administration is prepared using an excipient granulation according to the present invention.

### BRIEF DESCRIPTION OF THE DRAWINGS

Those skilled in the art will understand that the drawings described herein are for illustration purposes only. The drawings are not intended to limit the scope of the present disclosure.

FIG. 1 shows an example of a particle size distribution of an excipient granulation provided by the present disclosure.

FIG. 2 shows an example of the reconstitution viscosity of solutions prepared using excipient granulations.

FIG. 3 shows an example of the reconstitution luminosity of solutions prepared using excipient.

### DETAILED DESCRIPTION

For purposes of the following detailed description, it is to be understood that embodiments provided by the present disclosure may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

“Immediate release” refers to a composition that releases at least 80% of the constituents within 1 hour when tested in a dissolution apparatus 2 according to USP 38 in a 0.1 N HCl dissolution medium at a temperature of 37° C. and a paddle speed of 75 rpm. An immediate release composition or formulation can release substantially all of a pharmaceutically active ingredient into the gastrointestinal tract of a patient within less than 1 hour following oral administration, such as within less than 50 minutes, within less than 40 minutes, within less than 30 minutes, within less than 20 minutes, or within less than 10 minutes following oral administration. For example, an immediate release dosage form can release greater than 90%, greater than 95%, or greater than 98% of the constituents of the pharmaceutical composition into the gastrointestinal tract within less than 1 hour, such as within less than 50 minutes, less than 40 minutes, less than 30 minutes, less than 20 minutes, or less than 10 minutes, following oral administration. Immediate release pharmaceutical compositions can be useful for administering pharmaceutically active ingredients that are absorbed into the systemic circulation from the upper portion of the gastrointestinal tract.

“Modified release” pharmaceutical compositions and formulations can include controlled release formulations, delayed release formulations, extended-release formulations, sustained release formulations, timed release formulations, pulsatile release formulations, and pH-dependent release formulations. These formulations are intended to release a pharmaceutically active ingredient from the pharmaceutical composition at a desired rate and/or at a desired time following oral administration by a patient and/or at a certain location or locations within the gastrointestinal tract and/or at a certain pH within the gastrointestinal tract. The United States Pharmacopeia (USP) defines a modified release system as one in which the time course or location of drug release or both, are chosen to accomplish objectives of therapeutic effectiveness or convenience not fulfilled by immediate release dosage forms. A modified release oral dosage form can include extended release and delayed-release components. A delayed release dosage form is one that releases a drug all at once at a time other than promptly after administration. A modified release formulation can include delayed-release using enteric coatings, site-specific or timed release such as for colonic delivery, extended-release including, for example, formulations capable of providing zero-order, first-order, or biphasic release profiles, and programmed release such as pulsatile and delayed extended release.

“Patient” refers to a mammal, for example, a human.

“Pharmaceutically acceptable” refers to approved or approvable by a regulatory agency of the Federal or a state government or listed in the U.S. Pharmacopoeia (USP) or other generally recognized pharmacopoeia for use in animals, and more particularly in humans.

“Curing” a disease refers to eliminating the disease or disorder or eliminating a symptom of the disease or disorder.

“Treating” or “treatment” of a disease or disorder refers to reducing the severity of one or more clinical symptom of the disease or disorder, delaying the onset of one or more clinical symptoms of the disease or disorder, and/or mitigating one or more clinical symptoms of the disease or disorder. “Treating” or “treatment” of a disease or disorder refers to inhibiting the disease or disorder or one or more clinical symptoms of the disease or disorder, arresting the development of the disease or disorder or one or more clinical symptoms of the disease or disorder, relieving the disease or disorder or one or more clinical symptoms of the disease or disorder, causing the regression of the disease or disorder or one or more clinical symptoms of the disease or disorder, and/or stabilization of the disease or disorder or one or more clinical symptoms of the disease or disorder, “Treating” or “treatment” of a disease or disorder refers to producing a clinically beneficial effect without curing the underlying disease or disorder.

Reconstitution viscosity or the viscosity of an aqueous solution prepared using excipient granules can be determined using a Brookfield digital viscometer Model LVDV-1+ equipped with a Brookfield SC4-18 spindle at a spindle speed of 30 rpm at a temperature of 23° C. Excipient granules such as 1.2 g of the excipient granules can be added to water such as 40 mL of water in a 50 mL beaker and mixed for a specified time such as for 30, 60, 90, 120, 150, or 180 seconds. Mixing can be stopped and the viscosity measured.

Bulk density can be determined according to USP <616>, Method 1.

Tapped bulk density can be determined according to USP <616>.

The Hausner Ratio can be determined according to USP <1174>. The Hausner ratio is the ratio of the tapped density to the bulk density.

The compressibility index is the 100×[(tapped density−bulk density)/tapped density].

A particle size distribution or the average particle size of a granulation can be determined by laser diffraction or by sieve analysis.

The Flodex value can be determined according to USP <1174>.

Luminosity can be determined using a Technical Color Solutions Datacolor 650™ instrument.

Friability can be measured using a sonic sifter. Friability is defined as the number of microparticles having a diameter less than 75 μm that are generated by subjecting a granulation to a sonic sifter operated at a vibration amplitude of 8 corresponding to 3,600 sonic energy pulses per minute for at least 2 minutes.

A “pharmaceutical granulation” refers to a plurality of pharmaceutical granules where the pharmaceutical granules comprise an active pharmaceutical ingredient.

An “excipient granulation” refers to a plurality of excipient granules where the excipient granules do not comprise an active pharmaceutical ingredient.

“Pharmaceutical composition” refers to a composition comprising an excipient granulation and an active pharmaceutical ingredient.

“Oral pharmaceutical composition” refers to a pharmaceutical composition that is intended to be orally ingested by a patient.

Reference is now made to excipient granulations, excipient granules, pharmaceutical compositions comprising excipient granules, and uses of excipient granules. The disclosed excipient granulations, excipient granules, pharmaceutical compositions and uses are not intended to be limiting of the claims. To the contrary, the claims are intended to cover all alternatives, modifications, and equivalents.

An excipient granulation provided by the present disclosure can improve the palatability of an orally administered pharmaceutical granulation. Palatability can be defined as the overall appreciation of an oral medicinal product in relation to its smell, taste, aftertaste, and texture or feeling in the mouth. Palatability includes the overall acceptance of the taste, flavor, smell, does volume or size and texture of a medicine to be administered to the mouth and/or to be swallowed.

An excipient granulation provided by the present disclosure can increase the viscosity of an orally administered solution comprising a pharmaceutical granulation and thereby reduce the coarse or rough feeling in the mouth.

To enhance the convenience during use, an excipient granulation can provide a viscous solution capable of suspending a pharmaceutical granulation within 30 seconds after being combined with water.

A suitable solution viscosity for oral administration can be determined by a number of factors such as the density of the pharmaceutical granulation, the average size of the granules, the size distribution of the granules, and the average volume of the granules.

Excipient granules provided by the present disclosure can be particularly useful with high dose drugs. High dose drugs can require that large tablets or multiple tablets be administered. The inconvenience and/or discomfort of administering high dose drugs in the form of tablets can be avoided by using a pharmaceutical granulation. However, high dose granulations can produce a coarse or rough sensation in the mouth.

For convenience, it is desirable that a viscous solution prepared using an excipient granulation provided by the present disclosure be drinkable. A drinkable solution can have a viscosity, for example, from 50 cP to 600 cP at a temperature from 20° C. to 25° C., such as 23° C., determined using a Brookfield Digital Viscometer Model LVDV-I+ equipped with a Brookfield SC4-18 spindle at a spindle speed of 30 rpm.

However, excipient granules can also be used to provide higher viscosity pharmaceutical compositions that are more appropriately eaten. For example, a pharmaceutical composition can have a viscosity greater than 1,000 cP at a temperature from 20° C. to 25° C., such as 23° C., determined using a Brookfield Digital Viscometer Model LVDV-I+ equipped with a Brookfield SC4-18 spindle at a spindle speed of 30 rpm.

In an oral pharmaceutical composition provided by the present disclosure, the oral pharmaceutical composition can have a viscosity, for example, from 60 cP to 600 cP, such as from 200 cP to 600 cP, of from 300 cP to 600 cP, after 30 seconds following mixing with 40 mL of water at 23° C., determined using a Brookfield Digital Viscometer Model LVDV-I+ equipped with a Brookfield SC4-18 spindle at a spindle speed of 30 rpm.

In an oral pharmaceutical composition provided by the present disclosure, the oral pharmaceutical composition can have a viscosity, for example, from 200 cP to 1,100 cP, such as from 400 cP to 1,000 cP, or from 600 cP to 1,000 cP, after 60 seconds following mixing with 40 mL of water at 23° C., determined using a Brookfield Digital Viscometer Model LVDV-I+ equipped with a Brookfield SC4-18 spindle at a spindle speed of 30 rpm.

An oral pharmaceutical composition can have a viscosity similar to the viscosity of a drinkable yogurt product.

Intended objectives of an excipient granulation provided by the present disclosure include, for example, (1) to provide an oral pharmaceutical composition having a viscosity from 50 cP to 1,000 cP such as from 50 cP to 800 cP, from 50 cP to 600 cP, or from 50 cP to 500 cP, following mixing with an aqueous solution at 23° C. in less than 90 seconds; (2) to provide an average excipient granule size similar to that of a pharmaceutical granulation; (3) to provide excipient granules that are physically robust to storage and transportation; and/or (4) to provide a homogeneous granule composition such that the excipient granules and other granules such as pharmaceutical granules in the pharmaceutical composition do not segregate. It is desirable that the excipient granules and other granules in the composition remain homogeneously dispersed to facilitate the ability of the reconstituted solution and/or suspension to be homogeneous.

Additional intended objectives can include (1) taste enhancement; (2) pH maintenance; (3) rapid dissolution, and (4) low wt % or low volume.

An excipient granulation provided by the present disclosure comprises a plurality of excipient granules. An excipient granulation provided by the present disclosure can comprise pharmaceutically acceptable excipients.

When combined with water, an excipient granulation provides a viscous aqueous solution. The viscosity of the viscous aqueous solution can be selected to improve the palatability of a pharmaceutical composition comprising a pharmaceutical granulation dispersed and suspended in the viscous solution. A suitable viscosity effective in improving the palatability of a pharmaceutical granulation can depend, for example, on the average size of the pharmaceutical granules, the size distribution of the pharmaceutical granules, the density of the pharmaceutical granules, the volume percent of the pharmaceutical granules in the viscous aqueous solution, or a combination of any of the foregoing.

An excipient granulation provided by the present disclosure can be configured to provide a viscosity, when mixed with 40 mL of water at 23° C., for example, from 20 cP to 100 cP, determined using a Brookfield Digital Viscometer Model LVDV-I+ equipped with a Brookfield SC4-18 spindle at a spindle speed of 30 rpm. For example, when from 1 g to 2 g pf the excipient granulation is mixed with 40 mL of water at 23° C. in the amount of from 1 g to 3 g, from 20 cP to 100 cP, determined using a Brookfield Digital Viscometer Model LVDV-I+ equipped with a Brookfield SC4-18 spindle at a spindle speed of 30 rpm.

An excipient granule provided by the present disclosure can comprise a viscosifying agent, a disintegrant, and one or more additional excipients.

An excipient granule provided by the present disclosure can comprise an amount of each of the excipients suitable to accomplish an intended purpose of the excipient granulation such as, for example, to improve the palatability of a pharmaceutical granulation.

When combined with an aqueous solution an excipient granulation can provide a viscous solution comprising pharmaceutical granules dispersed and suspended in the viscous solution that are palatable for oral ingestion. The additional excipients of an excipient granulation provided by the present disclosure can be selected, for example, to reduce the sensation of granularity of the pharmaceutical granulation and to enhance the taste of the pharmaceutical composition. The additional excipients can be selected to adjust the pH of the pharmaceutical composition and to enhance the storage stability of the excipient granules and/or pharmaceutical granules.

An excipient granule provided by the present disclosure can comprise, for example, a viscosifying agent, a disintegrant, and one or more additional excipients. The one or more additional excipients is in addition to the viscosifying agent and the disintegrant.

An excipient granule provided by the present disclosure can comprise a viscosifying agent or a combination of viscosifying agents. A viscosifying agent can be a pharmaceutically acceptable viscosifying agent.

A viscosifying agent can be used to increase the viscosity of a liquid composition such as an aqueous composition including an oral pharmaceutical composition.

Suitable viscosifying agents include, for example, plant extracts such as a starch, apectin, an alginate, carrageenan, acacia gum, tragacanth, and cellulose; modified polysaccharides such as modified starches, amidated pectin, propylene glycol alginate; cellulose ethers such as microcrystalline cellulose, hypromellose, hydroxypropyl cellulose, and carboxymethyl cellulose; animal extracts such as gelatin, chitosan, and caseinates; fermentation products such as xanthan gum, gellan gum, pullulan, and dextran; seed polysaccharides such as guar gum and locust bean extract; and a combination of any of the foregoing.

A viscosifying agent can comprise, for example, a hydrocolloid gum including plant-derived hydrocolloids such as pectin, agar, alginates, acacia, tragacanth, Karaya gum, guar gum, starches, cellulose and locust bean; hydrocolloid products of fermentation such as xanthan gum, dextran, gellan gum, and pullulan; semi-synthetic hydrocolloids including modified starches, cellulose ethers such as methylcellulose, hypromellose, ethylcellulose, hydroxypropylcellulose and hydroxyethylcellulose), amidated pectin and propylene glycol alginate; animal derived hydrocolloids such as gelatin, chitosan and caseinates; and a combination of any of the foregoing.

A viscosifying agent can include aluminum silicates, such as bentonite, hectorite, and magnesium aluminum silicate.

A viscosifying agent can comprise a carbomer.

A viscosifying agent can comprise a polyol, a sugar, an oligosaccharide such as sorbitol, liquid maltitol, sucrose, fructose, dextrose, maltodextrin, and polydextrose.

Other suitable viscosifying agents include polyethylene glycol, polyethylene oxide, colloidal silicon dioxide, silicones, glycerin, fatty acids and hard fats.

A viscosifying agent can include vinyl polymers such as water-soluble polyvinylpyrrolidones such as povidone and crospovidone having an average molecular weight from 2,500 g/mol to 1,250,000 g/mol; ethylene-vinyl acetates; a polyvinyl alcohol having for example a degree of hydrolysis greater than 88% and a molecular weight from 20,000 g/mole to 100,000 g/mol.

Suitable viscosifying agents include, for example, guar gum, xanthan gum, sodium carboxymethyl cellulose, and combinations of any of the foregoing.

A viscosifying agent can be in the form of particles, where the particles have an average particle size of less than 75 μm, less than 50 μm, less than 35 μm, or less than 25 μm. Particles of a viscosifying agent can have an average particle size, for example, from 10 μm to 75 μm, from 20 μm to 70 μm, from 25 μm to 65 μm, or from 25 μm to 45 μm, wherein particle size is determined by sieve analysis or by laser diffraction. A viscosifying agent such as xanthan gum can have an average particle size of less than 35 μm.

A viscosifying agent can comprise xanthan gum such as Xnatural® 75 available from CP Kelco.

A viscosifying agent such as xanthan gum can have a particle size, for example, of not less than 100% through an 80 mesh (180 μm) screen, such as not less than 92% through a 200 mesh (75 μm) screen as determined using screen sieving. A viscosifying agent such as xanthan gum can have a loss on drying (LOD), for example, of not more than 15%. A viscosifying agent such as xanthan gum can provide a viscosity, for example, of from 1,200 mPaxs to 1,600 mPa×s at 1 wt % of the viscosifying agent in a 1% KCL solution at 60 rpm as determined using transmittance. A viscosifying agent such as xanthan gum can provide a viscosity ratio, for example, of from 1.02 to 1.45. A viscosifying agent such as xanthan gum can provide a solution pH, for example, from 6.0 to 8.0 at 1 wt % of the viscosifying agent in distilled water as determined using transmittance.

An excipient granule can comprise, for example, from 1 wt % to 40 wt % of a viscosifying agent, from 1 wt % to 35 wt %, from 1 wt % to 30 wt %, from 1 wt % to 20 wt %, from 5 wt % to 15 wt %, or from 8 wt % to 12 wt %, wherein wt % is based on the total weight of the excipient granule.

An excipient granule provided by the present disclosure can comprise a disintegrant or a combination of disintegrants. A disintegrant can be a pharmaceutically acceptable disintegrant.

A disintegrant facilitates rapid disintegration or breakdown and dissolution of the excipient granules following mixing with an aqueous solution at a temperature from 20° C. to 25° C. such as 23° C.

It can be desirable that the excipient granules completely dissolve in water in less than 30 seconds, in less than 60 seconds, in less than 90 seconds, or within less than 120 seconds following mixing in an aqueous solution at a temperature from 20° C. to 25° C. such as 23° C.

A disintegrant can comprise, for example, crospovidone, croscarmellose sodium, low-substituted hydroxypropyl cellulose, sodium starch glycolate, chitosan hydrochloride, corn starch and pregelatinized starch, calcium alginate & calcium sodium alginate, docusate sodium, microcrystalline cellulose, hydroxypropyl starch, magnesium aluminum silicate, methylcellulose, sodium alginate, starch, pregelatinized starch, calcium carboxymethylcellulose, calcium cellulose glycolate, carmellose calcium, powdered cellulose, or a combination of any of the foregoing.

Examples of suitable disintegrants include crospovidone, croscarmellose sodium, sodium starch glycolate, soy polysaccharide, crosslinked alginic acid, gellan gum, xanthan gum, calcium silicate, and crosslinked polyacrylate.

An excipient can comprise crospovidone such as crospovidone CL-M available from BASF.

A suitable crospovidone disintegrating agent can have a pH value from 5 to 8 as a 1 wt % solution in water at a temperature from 20° C. to 25° C. such as 23° C.; a bulk density from 0.25 g/cm³ to 0.45 g/cm³; and/or an average particle size from 20 μm to 40 μm.

An excipient granule can comprise, for example, from 1 wt % to 40 wt % of a disintegrant, from 1 wt % to 30 wt %, from 1 wt % to 25 wt %, from 1 wt % to 20 wt %, from 5 wt % to 20 wt %, from 7 wt % to 17 wt %, or from 10 wt % to 14 wt % of a disintegrant, wherein wt % is based on the total weight of the excipient granule.

An excipient granule provided by the present disclosure can comprise one or more additional excipients. An additional excipient can be a pharmaceutically acceptable excipient.

Examples of suitable excipients include preservatives, antioxidants, adsorbents, acidifying agents, alkalinizing agents, buffering agents, chelating agents, colorants, clarifying agents, emulsifying agents, flavorings, water-soluble polymers, diluents, binders, lubricants, wetting agents, surfactants, glidants, and filler.

The one or more additional excipients can comprise a diluent, a binder, a flavoring agent, a buffer, an acid, a sweetening agent, a filler, or a combination of any of the foregoing.

An excipient granule provided by the present disclosure can comprise, for example, from 40 wt % to 98 wt % of an additional excipient or combination of additional excipients, not including the viscosifying agent and the dispersant. An excipient granule provided by the present disclosure can comprise, for example, from 40 wt % to 98 wt % of one or more additional excipients, from 50 wt % to 95 wt %, from 60 wt % to 90 wt %, from 70 wt % to 85 wt %, or from 75 wt % to 85 wt % of one or more additional excipients, where wt % is based on the total weight of the excipient granule.

An excipient granule provided by the present disclosure can comprise, for example, less than 95 wt % of an additional excipient, less than 90 wt %, less than 80 wt %, less than 70 wt %, or less than 60 wt % of one or more additional excipients, where wt % is based on the total weight of the excipient granule. An excipient granule provided by the present disclosure can comprise, for example, greater than 40 wt % of one or more additional excipients, greater than 50 wt %, greater than 60 wt %, greater than 70 wt %, greater than 80 wt %, or greater than 90 wt % of one or more additional excipients, where wt % is based on the total weight of the excipient granule.

An excipient granule can comprise a diluent or combination of diluents. A diluent can be a pharmaceutically acceptable diluent.

A diluent can provide bulk to the excipient granule, enhance flow properties, and facilitate processing.

Examples of suitable diluents include microcrystalline cellulose, powdered cellulose, anhydrous lactose, lactose monohydrate, spray-dried lactose, mannitol, starch, pregelatinized starch, maize starch, corn starch, sorbitol, sucrose, compressible sugar, confectioner's sugar, sugar spheres, dextrates, dextrin, dextrose, dibasic calcium phosphate, anhydrous calcium phosphate, calcium carbonate, maltose, maltodextrin, kaolin, dibasic calcium phosphate, dihydrate calcium phosphate, tribasic calcium phosphate, calcium sulfate, cellaburate, calcium lactate, cellulose acetate, silicified microcrystalline cellulose, cellulose acetate, corn syrup, pregelatinized starch and corn starch, corn syrup solids, erythritol, ethylcellulose, ethyl acrylate and methyl methacrylate copolymer dispersion, fructose, isomalt, alpha-lactalbumin, lactitol, magnesium carbonate, magnesium oxide, methacrylic acid acid ethyl acrylate copolymer, methacrylic acid and methyl methacrylate copolymer, polydextrose, sodium chloride, simethicone, pregelatinized modified starch, pea starch, hydroxypropyl pea starch, pregelatinized hydroxypropyl pea starch, potato starch, hydroxypropyl potato starch, pregelatinized hydroxypropyl potato starch, starch, tapioca, wheat starch, hydrogenated starch hydrolysate, pullulan, talc, amino methacrylate copolymer, trehalose, xylitol, and a combination of any of the foregoing.

Examples of suitable diluents include lactose, microcrystalline cellulose, starch, calcium phosphate such as anhydrous dibasic calcium phosphate, hydrated dibasic calcium phosphate, and tribasic calcium phosphate, calcium carbonate, maltodextrin, mannitol, sorbitol, and sodium chloride.

A diluent can comprise anhydrous dibasic calcium phosphate.

An anhydrous dibasic calcium phosphate can be A-Comprez® available from Markan Global Enterprises.

An excipient granule can comprise, for example, from 1 wt % to 40 wt %, from 10 wt % to 30 wt %, from 15 wt % to 25 wt %, or from 17 wt % to 23 wt % of a diluent such as anhydrous dibasic calcium phosphate, where wt % is based on the total weight of the excipient granule.

An excipient granule provided by the present disclosure can comprise a binder or a combination of binders. A binder can be a pharmaceutically acceptable binder.

A binder promotes cohesion of the of the constituents of the excipient granule.

Examples of suitable binders include natural binders such as starch, pregelatinized starchy, sodium alginate, and gelatin; synthetic and semi-synthetic binders such as polyvinylpyrrolidone, methylcellulose, hydroxypropyl methyl cellulose, polymethacrylates, sodium carboxy methyl cellulose, polyethylene glycol, and methylcellulose; saccharides such as lactose, sucrose, starches, cellulose and modified cellulose such as microcrystalline cellulose and cellulose ethers such as hydroxypropyl cellulose, and sugar derivatives such as sorbitol, xylitol, and mannitol.

Other examples of suitable binders include acacia, copovidone, carbomer, corn starch and pregelatinized starch, calcium carboxymethylcellulose, calcium cellulose glycolate, carmellosum calcium, carboxymethylcellulose sodium, carmellose sodium, Ceratonia, chitosan hydrochloride, dextrates, dextrin, ethylcellulose, liquid glucose, guar gum, hydroxyethyl cellulose, hydroxyethylmethyl cellulose, hydroxypropyl cellulose, low-substituted hydroxypropyl cellulose, hydroxypropyl starch, hydroxypropyl methylcellulose, inulin, magnesium aluminum silicate, maltodextrin, methylcellulose, polyethylene glycol, polyethylene oxide, povidone, sodium alginate, starch, pregelatinized starch, sucrose, compressible sugar, zein, gelatin, polymethylmethacrylates, sorbitol, glucose, and sodium alginate.

Examples of suitable binders include hydroxypropyl cellulose, polyvinyl alcohol, hydroxypropylmethyl cellulose, hydroxypropylethyl cellulose, polyvinylpyrrolidone, or polyethylene glycol.

A binder can comprise a polyvinylpyrrolidone.

A suitable polyvinylpyrrolidone can be characterized by an average molecular weight, for example, of 58,000 Daltons, where the average molecular weight is determined by column chromatography.

A suitable polyvinylpyrrolidone can have a viscosity, for example, of 2.5 cP in a 5% aqueous solution at 25° C.

A suitable polyvinylpyrrolidone can have an average particle size, for example, of 80 μm.

A suitable polyvinylpyrrolidone can have a bulk density, for example, of 0.36 g/cm³ and/or a tapped density of 0.47 g/cm³.

An excipient granule can comprise, for example, from 1 wt % to 30 wt %, from 2 wt % to 25 wt % from 5 wt % to 20 wt %, or from 7 wt % to 13 wt % of a binder such as polyvinylpyrrolidone, where wt % is based on the total weight of the excipient granule.

An excipient granule provided by the present disclosure can comprise a flavoring agent or a combination of flavoring agents. A flavoring agent can be a pharmaceutically acceptable flavoring agent.

A flavoring agent can render a pharmaceutical composition more palatable and can mask an unpleasant taste of an active pharmaceutical ingredient. The nature of the unpleasant taste to be masked can determine the selection of a suitable flavoring agent.

Examples of suitable flavoring agents include vanillin, peppermint flavor powder, berry flavor powder, strawberry flavor powder, orange flavor powder, lemon flavor powder, orange essence, ethyl maltol, eucalyptus oil, isobutyl alcohol, sodium succinate, adipic acid, almond oil, anethole, benzaldehyde, denatonium benzoate, ethyl acetate, ethyl vanillin, ethylcellulose, fructose, fumaric acid, l-glutamic acid, hydrochloride, lactitol, leucine, malic acid, maltol, menthol/racementhol, methionine, methyl salicylate, monosodium glutamate, peppermint oil, strawberry flavor, liquid, peppermint spirit, racemethionine, rose oil, rose water, stronger, sodium acetate, sodium lactate solution, tartaric acid, thymol, fumaric acid, inulin, isomalt, and neohesperidin dihydrochalcone.

A flavoring agent can comprise a citric fruit flavoring agent, a berry flavoring agent, a punch flavoring agent, or a combination of any of the foregoing.

An excipient granule provided by the present disclosure can comprise, for example, from 1 wt % to 40 wt % of a flavoring agent, from 5 wt % to 35 wt %, from 10 wt % to 35 wt % from 15 wt % to 35 wt %, from 20 wt % to 30 wt %, or from 22 wt % to 28 wt %, of a flavoring agent, where wt % is based on the total weight of the excipient granule.

An excipient granule provided by the present disclosure can comprise a buffer or a combination of buffers. A buffer can be a pharmaceutically acceptable buffer.

Examples of suitable buffers include sodium citrate dihydrate, citric acid anhydrous, sodium phosphate dibasic, and sodium phosphate monobasic, potassium metaphosphate, potassium phosphate, monobasic sodium acetate, and anhydrous sodium citrate.

A buffer can comprise sodium citrate dihydrate.

An excipient granule can comprise, for example, from 0.1 wt % to 10 wt % of a buffer, from 0.5 wt % to 8 wt %, from 0.5 wt % to 6.5 wt %, from 1 wt % to 5 wt %, or from 2 wt % to 4 wt % of a buffer such as sodium citrate dihydrate, where wt % is based on the total weight of the excipient granule.

An excipient granule provided by the present disclosure can comprise an acidifying agent or a combination of acidifying agents. An acidifying agent can be a pharmaceutically acceptable acidifying agent.

Examples of suitable acidifying agent include anhydrous sodium citrate, fumaric acid, hydrochloric acid, and nitric acid.

An excipient granule can comprise, for example, from 0.1 wt % to 10 wt % of an acid, from 0.5 wt % to 8 wt %, from 1 wt % to 7 wt %, from 2 wt % to 6 wt %, or from 3 wt % to 5 wt % of an acidifying agent such as anhydrous sodium citrate, where wt % is based on the total weight of the excipient granule.

An excipient granule provided by the present disclosure can comprise a sweetening agent or a combination of sweetening agents. A sweetening agent can comprise a pharmaceutically acceptable sweetening agent.

Examples of suitable sweetening agents include sucralose, saccharin sodium, neotame, sucrose, acesulfame potassium, aspartame, aspartame acesulfame, corn syrup, corn syrup solids, dextrates, dextrose, dextrose excipient, erythritol, fructose, galactose, glucose, glycerin, inulin, invert sugar, isomalt, lactitol, maltitol, maltose, mannitol, saccharin, saccharin calcium, sorbitol, starch hydrolysate, hydrogenated, sugar, compressible, sugar, confectioner's, tagatose, trehalose, and xylitol.

Examples of suitable sweetening agents include mannitol, sucralose, aspartame, dextrose, glycerin, saccharin sodium, sorbitol, and sucrose.

A sweetening agent can comprise sucralose.

An excipient granule can comprise, for example, from 0.1 to 10 wt % of a sweetening agent, from 0.1 wt % to 7 wt %, from 0.1 wt % to 6 wt %, from 1 wt % to 5 wt %, or from 2 wt % to 4 wt % of a sweetening agent such as sucralose, where wt % is based on the total weight of the excipient granule.

An excipient granule provided by the present disclosure can comprise a filler or combination of filler. A filler can be a pharmaceutically acceptable filler.

Examples of suitable filler include microcrystalline cellulose, powdered cellulose, anhydrous lactose, lactose monohydrate, spray-dried lactose, mannitol, starch, pregelatinized starch, maize starch, corn starch, sorbitol, sucrose, compressible sugar, confectioner's sugar, sugar spheres, dextrate, dextrin, dextrose, calcium phosphate, dibasic, anhydrous, calcium carbonate, maltose, maltodextrin, kaolin, calcium phosphate, dibasic, dihydrate, tribasic calcium phosphate, calcium sulfate, cellaburate, calcium lactate, cellulose acetate, silicified microcrystalline cellulose, cellulose acetate, corn syrup, pregelatinized starch and corn starch, corn syrup, solids, erythritol, ethylcellulose, ethyl acrylate and methyl methacrylate copolymer dispersion, fructose, isomaltose, alpha-lactalbumin, lactitol, magnesium carbonate, magnesium oxide, methacrylic acid and ethyl acrylate copolymer, methacrylic acid and methyl methacrylate copolymer, polydextrose, sodium chloride, simethicone, pregelatinized modified starch, starch, pea, hydroxypropyl pea starch, starch, pregelatinized hydroxypropyl pea, potato starch, starch, hydroxypropyl potato, pregelatinized hydroxypropyl potato starch, starch, tapioca, wheat starch, starch hydrolysate, hydrogenated, pullulan, talc, amino methacrylate copolymer, trehalose, xylitol, and combinations of any of the foregoing.

Examples of suitable filler include starch 1500, starch 1500 (gelatinized), microcrystalline cellulose, calcium phosphate powder, dicalcium phosphate dihydrate, mannitol, or dicalcium phosphate powder, lactose, calcium carbonate, calcium sulfate, compressible sugars, dextrates, dextrin, dextrose, kaolin, magnesium carbonate, magnesium oxide, maltodextrin, powdered cellulose, sucrose, and combinations of any of the foregoing.

A filler can comprise microcrystalline cellulose such as Avicel® PH-101 or Celphere® CP-305 available from Asahi-Kasei.

An excipient granule provided by the present disclosure can comprise, for example, from 1 wt % to 20 wt % of a filler such as microcrystalline cellulose, from 5 wt % to 15 wt %, or from 7 wt % to 13 wt % of a filler such as microcrystalline cellulose, where wt % is based on the total weight of the excipient granule.

An excipient granule provided by the present disclosure can comprise, for example, a viscosifying agent, a disintegrant, and one or more additional excipients where the one or more additional excipients can comprise a filler or combination of fillers. An excipient granule can comprise, for example, from 40 wt % to 98 wt % of a filler, from 50 wt % to 90 wt %, or from 60 wt % to 80 wt % of a filler, where wt % is based on the total weight of the excipient granule. An excipient granule can comprise, for example, less than 90 wt % of a filler, less than 80 wt %, less than 70 wt %, less than 60 wt %, less than 50 wt % of a filler, where wt % is based on the total weight of the excipient granule. An excipient granule can comprise, for example greater than 40 wt % of a filler, greater than 50 wt %, greater than 60 wt %, greater than 70 wt %, greater than 80 wt %, or greater than 90 wt % of a filler, where wt % is based on the total weight of the excipient granules.

An excipient granule provided by the present disclosure can comprise a pharmaceutically acceptable glidant or combination of pharmaceutically acceptable glidants. Examples of suitable pharmaceutically acceptable glidants include magnesium stearate, magnesium silicate, calcium stearate, sodium lauryl sulphate, sodium stearyl fumarate, magnesium lauryl sulphate, stearic acid, calcium stearate, glyceryl behenate, behenoyl polyoxylglycerides, glyceryl dibehenate, lauric acid, glyceryl monostearate, glyceryl tristearate, myristic acid, palmitic acid, poloxamer, polyethylene glycol, polyethylene glycol 3350, polysorbate 20, polyoxyl 10 oleyl ether, polyoxyl 15 hydroxystearate, polysorbate 40, polyoxyl 20 cetostearyl ether, polyoxyl 40 stearate, polysorbate 60, polysorbate 80, potassium benzoate, sodium benzoate, sorbitan monolaurate, sorbitan monooleate, sodium stearate, sorbitan monopalmitate, sorbitan monostearate, zinc stearate, sorbitan sesquioleate, sorbitan trioleate, and talc.

An excipient granule provided by the present disclosure can comprise a pharmaceutically acceptable lubricant or combination of pharmaceutically acceptable lubricants. Examples of suitable pharmaceutically acceptable lubricants include colloidal silicon dioxide, talc, tribasic calcium phosphate, calcium silicate, cellulose, powdered, magnesium oxide, sodium stearate, magnesium silicate, silica, dental-type, magnesium trisilicate, and hydrophobic colloidal silica.

An excipient granule provided by the present disclosure can comprise a pharmaceutically acceptable alkalinizing agent or combination of pharmaceutically acceptable alkalinizing agents. Examples of suitable pharmaceutically acceptable alkalinizing agents include ammonium carbonate, diethanolamine, monoethaolamine, potassium hydroxide, sodium bicarbonate, sodium borate, sodium carbonate, sodium hydroxide, and trolamine.

An excipient granule provided by the present disclosure can comprise a pharmaceutically acceptable surfactant or combination of pharmaceutically acceptable surfactants. Examples of suitable pharmaceutically acceptable surfactants include behenoyl polyoxylglycerides, Polysorbate 20, Polysorbate 40, docusate sodium, Polysorbate 60, Polysorbate 80, benzalkonium chloride, caprylocaproyl polyoxylglycerides, cetylpyridinium chloride, lauroyl polyoxylglycerides, linoleoyl polyoxylglycerides, Octoxynol 9, oleoyl polyoxylglycerides, poloxamer, Polyoxyl 10 oleyl ether, Polyoxyl 15 hydroxystearate, Nonoxynol 9, Polyoxyl 20 cetostearyl ether, Polyoxyl 40 stearate, pullulan, polyoxyl lauryl ether, polyoxyl stearyl ether, sodium lauryl sulfate, sorbitan monolaurate, sorbitan monooleate, polyoxyl stearate, sorbitan monopalmitate, sorbitan monostearate, stearoyl polyoxylglycerides, sorbitan sesquioleate, sorbitan trioleate, and tyloxapol.

An excipient granule provided by the present disclosure can comprise a pharmaceutically acceptable coloring agent or combination of pharmaceutically acceptable coloring agents. Examples of suitable pharmaceutically acceptable coloring agents include caramel, ferric oxide, titanium dioxide, ferrosoferric oxide, aluminum oxide, FD & C Red #40/Allura Red AC, amaranth, FD & C Blue #1/Brilliant Blue FCF, canthaxanthin, carmine, carmoisine (Azorubine), curcumin (tumeric), FD & C Red #3/Erythrosine, Fast Green FCF, Green S (Lissamine Green), D & C Red #30/Helendon Pink, FD & C Blue #2/Indigo Carmine, iron oxide black, iron oxide red, D & C Red #7/Lithol Rubin BK, Patent Blue V, D & C Red F128/Phloxine B, Iron Oxide Yellow, D & C Red Fr27/Phloxine O, Ponceau 4R (Cochincal Red A), Quinoline Yellow WS, D & C Yellow #10, Riboflavin (Lactoflavin), FD & C Yellow #5/Tartrazine, and FD & C Yellow #6/Sunset Yellow FCF.

An excipient granule and excipient granulation provided by the present disclosure can comprise, for example, from 2 wt % to 22 wt % of a binder; from 10 wt % to 30 wt % of a diluent; from 1 wt % to 20 wt % of a filler; from 15 wt % to 35 wt % of a flavoring agent; from 0.5 wt % to 6.5 wt % of a buffer; from 0.5 wt % to 8 wt % of an acidifying agent; and/or from 0.1 wt % to 6 wt % of a sweetening agent, wherein wt % is based on the total weight of the excipient granule or excipient granulation.

An excipient granule and excipient granulation provided by the present disclosure can comprise, for example, from 1 wt % to 35 wt % of a viscosifying agent; and from 1 wt % to 25 wt % of a disintegrant; wherein wt % is based on the total weight of the excipient granule or excipient granulation.

An excipient granule and excipient granulation provided by the present disclosure can comprise, for example, from 5 wt % to 15 wt % of a binder; from 17 wt % to 27 wt % of a diluent; from 6 wt % to 16 wt % of a filler; from 20 wt % to 30 wt % of a flavoring agent; from 1 wt % to 5 wt % of a buffer; from 2 wt % to 6 wt % of an acidifying agent; and/or from 1 wt % to 5 wt % of a sweetening agent, wherein wt % is based on the total weight of the excipient granule or excipient granulation.

An excipient granule and excipient granulation provided by the present disclosure can comprise, for example, from 5 wt % to 15 wt % of a viscosifying agent; and from 7 wt % to 17 wt % of a disintegrant; wt % is based on the total weight of the excipient granules or excipient granulation.

An excipient granule and excipient granulation provided by the present disclosure can comprise, for example, the viscosifying agent comprises xanthan gum; the disintegrant comprises crospovidone; the binder comprises polyvinylpyrrolidone; the diluent comprises dicalcium phosphate; the filler comprises microcrystalline cellulose; the flavoring agent comprises a berry/punch flavoring agent; the buffer comprises sodium citrate dihydrate; the acidifying agent comprises citric acid anhydrous; and/or the sweetening agent comprises sucralose, wherein wt % is based on the total weight of the excipient granule or excipient granulation.

An excipient granulation provided by the present disclosure can have an average granule size, for example, from 100 μm to 600 μm, from 200 μm to 600 μm, from 100 μm to 550 μm, from 100 μm to 500 μm, from 200 μm to 500 μm or from 250 μm to 450 μm, where the average granule size is determined by sieve analysis or by laser diffraction.

An excipient granulation can have a particle size distribution characterized, for example, by 8.5 wt % greater than 35 mesh (500 μm), 39 wt % from 35-60 mesh (500-250 μm), 52.2% pan, less than 35 mesh (500 μm) 52.2 wt %; and the particle size is determined by sieve analysis or laser diffraction.

An excipient granulation (35-60 mesh fraction) can have an average bulk density, for example, from 0.5 g/mL to 0.9 g/mL, where the bulk density is determined using a bulk density cylinder.

An excipient granulation provided by the present disclosure can have a bulk density, for example, from 0.5 g/mL to 0.7 g/mL, from 0.6 g/mL to 0.9 g/mL, from 0.65 g/L to 0.85 g/mL, or from 0.7 g/mL to 0.8 g/mL, where the bulk density is determined according to USP <616>, Method 1.

An excipient granulation provided by the present disclosure can have an average tapped density, for example, from 500 g/mL to 900 g/mL, from 550 g/L to 850 g/mL, from 600 g/mL to 800 g/mL, or from 650 g/mL to 750 g/mL, where the average tapped density is determined according to USP <616>.

An excipient granulation provided by the present disclosure can have a Hausner ratio, for example, from 1.0 to 1.5, from 1.1 to 1.4, from 1.1 to 1.35, or from 1.1 to 1.25, where the average Hausner ratio is determined according to USP <1174>.

An excipient granulation provided by the present disclosure can have a compressibility index, for example, from 8 to 22, from 10 to 20, or from 12 to 18.

An excipient granulation provided by the present disclosure can have a Flodex value, for example, of less than 5 mm, less than 4 mm, less than 3 mm, or less than 2 mm, where the Flodex value is determined according to USP <1174>.

An excipient granulation provided by the present disclosure can have a friability, for example, of less than 2, or less than 1, where friability is determined using a sonic sifter.

An excipient granulation provided by the present disclosure can have a loss on drying, for example, from 1 wt % to 4 wt %, such as from 1.5 wt % to 3.5 wt %, or from 2 wt % to 3 wt %, where the loss on drying is determined by thermogravimetric analysis.

An excipient granulation when mixed with water at a concentration of from 0.01 g/mL to 0.04 g/mL can provide a solution having a viscosity of greater than 20 cP, greater than 30 cP, greater than 40 cP, greater than 50 cP, greater than 60 cP, greater than 70 cP, greater than 80 cP, greater than 90 cP, or greater than 100 cP, in less than 2 minutes with stirring, agitating, and/or shaking at a temperature from 20° C. to 25° C. For example, excipient granules when mixed with water at a concentration of from 0.01 g/mL to 0.04 g/mL can provide a solution can provide a solution having a viscosity from 40 cP to 80 cP in less than 30 seconds, in less than 60 seconds, in less than 90 seconds, or less than 120 seconds with stirring, agitating and/or shaking at a temperature from 20° C. to 25° C. such as 23° C. Viscosity can be determined using a Brookfield Digital Viscometer Model LVDV-I+ equipped with a Brookfield SC4-18 spindle at a spindle speed of 30 rpm.

Excipient granules provided by the present disclosure can be intended to provide a viscous solution within 90 seconds of being combined and mixed with an aqueous solution at a temperature from 20° C. to 25° C. such as a 23° C. The maximum viscosity of the aqueous solution can be, for example, from 10 cP to 200 cP, from 25 CP to 150 cP, from 50 cP to 100 cP, from 40 cP to 80 cP, or from 50 cP to 70 cP at a temperature from 20° C. to 25° C. such as 23° C.

An excipient granulation provided by the present disclosure can completely dissolve when mixed with an aqueous solution at a temperature of 20 C to 25° C. such as 23° C., for example, in less than 120 seconds, in less than 90 seconds, in less than 60 seconds, or less than 30 seconds, where completely dissolved is defined as the time from when the excipient granulation is combined with the aqueous solution to the time when the viscosity of the viscous solution is greater than 90% of the maximum viscosity.

A solution prepared by combining from 1 wt % to 5 wt % of an excipient granulation with from 95 wt % to 99 wt % water at a temperature from 20° C. to 25° C. can achieve a maximum viscosity, for example, within from 15 seconds to 120 seconds such as from 15 seconds to 60 seconds following mixing, where wt % is based on the total weight of the excipient granulation and the water. Mixing can be accomplished, for example, by stirring, agitating, and/or swirling the excipient granulation in water or a non-viscous aqueous solution.

A solution prepared by combining from 1 wt % to 5 wt % of the excipient granulation with from 95 wt % to 99 wt % water at a temperature from 20° C. to 25° C. such as 23° C. can provide a solution having a viscosity, for example, of from 25 cp to 200 cp.

An excipient granulation provided by the present disclosure, when reconstituted as a 1 w/v % solution in water at a temperature from 20° C. to 25° C. such as 23° C., can completely dissolve in less than 60 seconds or in less than 30 seconds.

An excipient granulation provided by the present disclosure, when reconstituted as a 1 w/v % solution in water at a temperature from 20° C. to 25° C. such as 23° C., can provide a solution having a pH, for example from 4.0 to 8.0, such as from 4.0 to 7.0, or from 4.0 to 5.0.

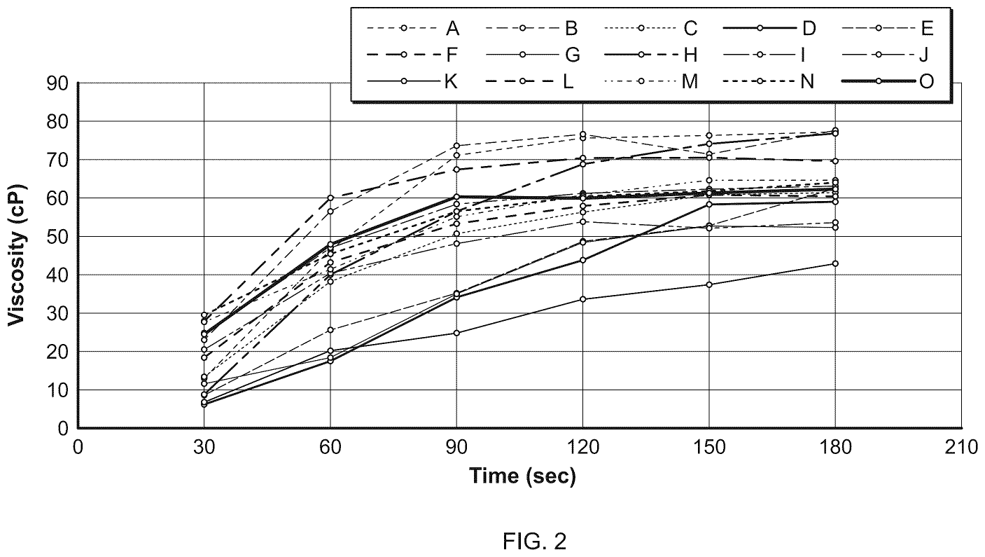

When 1.2 g of an excipient granulation provided by the present disclosure is reconstituted in 40 mL of water at a temperature from 20° C. to 25° C. such as 23° C., the resulting solution exhibits a viscosity vs. time profile as shown in FIG. 2.

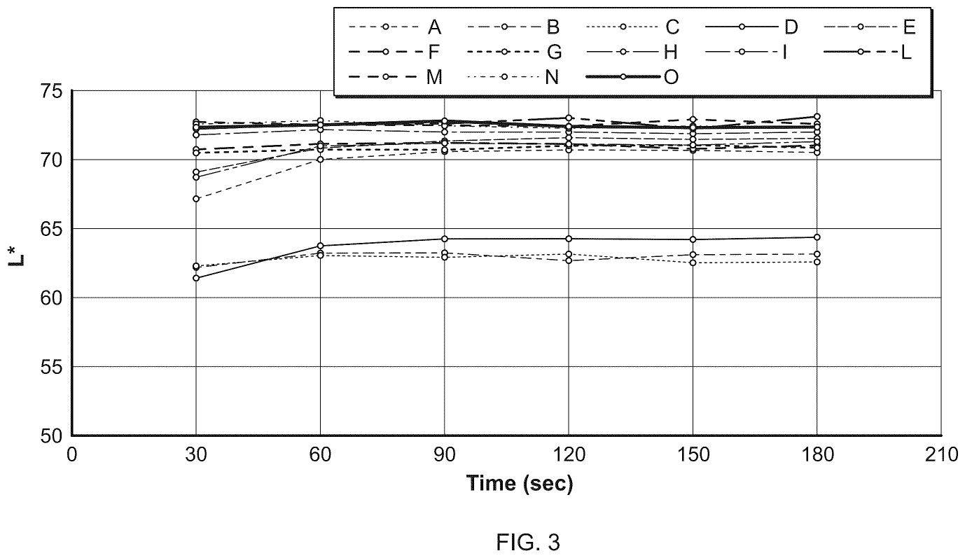

When 1.2 g of an excipient granulation provided by the present disclosure is reconstituted in 40 mL of water at a temperature from 20° C. to 25° C. such as 23° C., the resulting solution exhibits a luminosity vs. time profile as shown in FIG. 3.

An excipient granulation can be configured to provide a viscosity of from 20 cP to 100 cP when 1.2 gm of the excipient granulation is combined with 40 mL of water at a temperature from 20° C. to 25° C.

After mixing a solution of 1.2 g of an excipient granulation provided by the present disclosure and 40 mL of water at a temperature from 20° C. to 25° C. such as 23° C. for 90 seconds, the solution can have a viscosity within a range from 45 cP to 80 cP.

After mixing a solution of 1.2 g of an excipient granulation provided by the present disclosure and 40 mL of water at a temperature from 20° C. to 25° C. such as 23° C. for 60 seconds, the solution can have a viscosity within a range from 35 cP to 60 cP.

After mixing a solution of 1.2 g of an excipient granulation provided by the present disclosure and 40 mL of water at a temperature from 20° C. to 25° C. such as 23° C. for 30 seconds, the solution can have a viscosity within a range from 15 cP to 40 cP.

An excipient granulation provided by the present disclosure can provide a composition having a reconstitution pH of from 4 to 6.

A composition provided by the present disclosure can comprise an excipient granulation provided by the present disclosure and one or more additional constituents. For example, a composition can comprise an excipient granulation and an antistatic agent in the form of a powder to minimize agglomeration of the excipient granules during storage and/or to facilitate dispersion of the excipient granules when combined with a solution.

A composition provided by the present disclosure can comprise a pharmaceutical composition.

A pharmaceutical composition provided by the present disclosure can comprise an excipient granulation provided by the present disclosure and an active pharmaceutically ingredient.

A pharmaceutical composition can comprise an active pharmaceutical ingredient or a combination of active pharmaceutical ingredients.

An active pharmaceutical ingredient can comprise any suitable active pharmaceutical ingredient such as any active pharmaceutical ingredient that can be administered orally. An active pharmaceutical ingredient can be absorbed from the gastrointestinal tract into the systemic circulation.

An active pharmaceutical ingredient includes, for example, an active pharmaceutical ingredient as a free base, a pharmaceutically acceptable salt of the active pharmaceutical ingredient, a prodrug of the active pharmaceutical ingredient, a hydrate of the active pharmaceutical ingredient, or a combination of any of the foregoing.

An active pharmaceutical ingredient can be provided, for example, in the form of solution, a suspension, a powder, a granulation, or a tablet. The active pharmaceutical ingredient can be combined with one or more pharmaceutically acceptable excipients.

An active pharmaceutical ingredient can be soluble in an aqueous solution at a temperature from 20° C. to 25° C. or can be in the form of microparticles, granules, or powder, where the particles are not soluble in the aqueous solution or are sparingly soluble in the aqueous solution.

An active pharmaceutical ingredient in the form of a dry powder can comprise particles having an average size of less than 50 μm, less than 10 μm, or less than 1 μm.

A granulation comprising an active pharmaceutical ingredient is referred to as a pharmaceutical granulation.

A pharmaceutical composition provided by the present disclosure can comprise an excipient granulation and a pharmaceutical granulation.

Physical properties of an excipient granulation can be selected to minimize or prevent settling or segregation of a pharmaceutical composition comprising an excipient granulation and a pharmaceutical granulation. To facilitate the ability of the pharmaceutical granulation to become homogeneously dispersed in a viscous solution prepared using the excipient granulation, it can be desirable that the pharmaceutical composition comprising the excipient granulation and the pharmaceutical granulation be homogenously dispersed and remain homogenously dispersed during storage and transportation. Homogeneous dispersion of an excipient granulation and a pharmaceutical granulation of a pharmaceutical composition can be facilitated by having the physical properties of the excipient granulation and a pharmaceutical granulation be similar. For example, it can be desirable that certain physical properties such as, for example, the average particle size, the bulk density, the tapped density, the Hausner ratio, the compressibility index, the Flodex value, and/or the friability value of the excipient granulation and the pharmaceutical granulation be similar. For example, it can be desirable that a value of a physical property of an excipient granulation be within ±20%, within ±15%, within ±10%, within ±5%, or within less than ±2% the value of the corresponding property of a pharmaceutical granulation.

For a pharmaceutical granulation comprising more than one population of granulations such as an immediate release pharmaceutical granulation and a modified release pharmaceutical granulation it can be desirable that a value of a physical property of an excipient granulation be within ±20%, within 15%, within ±10%, within ±5%, or within less than ±2% the value of the corresponding property of each of the immediate release pharmaceutical granulation and the modified release granulation.

In a pharmaceutical composition provided by the present disclosure, an excipient granulation provided by the present disclosure can have an average granule size that is, for example, within ±20% of the average particle size of a pharmaceutical granulation with which the excipient granulation is combined, within ±15%, within ±10%, or within ±5% of the average particle size of a pharmaceutical granulation with which the excipient granulation is combined.

In a pharmaceutical composition provided by the present disclosure, an excipient granulation provided by the present disclosure can have an average bulk density that is, for example, within ±20% of the average bulk density of a pharmaceutical granulation with which the excipient granulation is combined, within ±15%, within ±10%, within ±5%, or within less than ±2% of the average bulk density of a pharmaceutical granulation with which the excipient granulation is combined.

In a pharmaceutical composition provided by the present disclosure, an excipient granulation provided by the present disclosure can have an average tapped density that is, for example, within ±20% of the average tapped density of a pharmaceutical granulation with which the excipient granulation is combined, within ±15%, within ±10%, within ±5%, or within less than ±2% of the average tapped density of a pharmaceutical granulation with which the excipient granulation is combined.

In a pharmaceutical composition provided by the present disclosure, an excipient granulation provided by the present disclosure can have a Hausner ratio that is, for example, within ±20% of the average Hausner ratio of a pharmaceutical granulation with which the excipient granulation is combined, within ±15%, within ±10%, within ±5%, or within less than ±2% of the Hausner ratio of a drug-containing granulation with which the excipient granulation is combined.

In a pharmaceutical composition provided by the present disclosure, an excipient granulation provided by the present disclosure can have a compressibility index that is, for example, within ±20% of the compressibility index of a pharmaceutical granulation with which the excipient granulation is combined, within ±15%, within ±10%, within ±5%, or within less than ±2% of the compressibility index of a pharmaceutical granulation with which the excipient granulation is combined.

In a pharmaceutical composition provided by the present disclosure, an excipient granulation provided by the present disclosure can have a Flodex value that is, for example, within ±20% of the Flodex value of a pharmaceutical granulation with which the excipient granulation is combined, within ±15%, within ±10%, or within ±5%, or within less than ±2% of the Flodex value of a pharmaceutical granulation with which the excipient granulation is combined.

In a pharmaceutical composition provided by the present disclosure, an excipient granulation provided by the present disclosure can have a friability that is, for example, within ±20% of the friability of a pharmaceutical granulation with which the excipient granulation is combined, within ±15%, within ±10%, or within ±5%, or within less than ±2% of the friability of a pharmaceutical granulation with which the excipient granulation is combined.

A pharmaceutical granulation provided by the present disclosure can comprise a plurality of pharmaceutical granules.

A pharmaceutical granule can comprise an active pharmaceutical ingredient and one or more pharmaceutically acceptable excipients.

A pharmaceutical granulation can comprise any suitable pharmaceutically active ingredient that is suitable for oral administration and that can be absorbed from the gastrointestinal tract into the systemic circulation. A pharmaceutical granulation can comprise one or more pharmaceutically acceptable excipients. The one or more pharmaceutically acceptable excipients can comprise any suitable pharmaceutically acceptable excipients. Examples of suitable pharmaceutically acceptable excipients include, for example, binders, fillers, dispersing agents, pH modifiers, etc.

A pharmaceutical granulation can comprise, for example, greater than 10 wt % of an active pharmaceutical ingredient, greater than 20 wt %, greater than 30 wt %, greater than 40 wt %, greater than 50 wt %, greater than 60 wt %, greater than 70 wt %, greater than 80 wt %, or greater than 90 wt % of an active pharmaceutical ingredient, where wt % is based on the total weight of the pharmaceutical granulation.

A pharmaceutical granulation can comprise, for example, from 1 wt % to 99 wt % of an active pharmaceutical ingredient, from 20 wt % to 99 wt %, from 50 wt % to 99 wt %, from 70 wt % to 99 wt %, from 70 wt % to 95 wt %, or from 70 wt % to 90 wt % of an active pharmaceutical ingredient, where wt % is based on the total weight of the pharmaceutical granulation.

A pharmaceutical granulation can comprise a pharmaceutically acceptable excipient or combination of pharmaceutically acceptable excipients.

A pharmaceutical granulation can comprise, for example, from less than 10 wt % of one or more pharmaceutically acceptable excipients, less than 20 wt %, less than 30 wt %, less than 40 wt %, less than 50 wt %, or less than 60 wt % of one or more pharmaceutically acceptable excipients where wt % is based on the total weight of the pharmaceutical granulation.

A pharmaceutical containing granulation can comprise the same or different excipient or combination of excipients as used in an excipient granulation.

A pharmaceutical granulation provided by the present disclosure can have an average granule size, for example, from 100 μm to 600 μm, from 200 μm to 600 μm, from 150 μm to 550, μm, from 200 μm to 500 μm, or from 250 μm to 450 μm, where the average granule size is determined by sieve analysis or laser diffraction.

A pharmaceutical granulation comprises a plurality of pharmaceutical granules comprising an active pharmaceutical ingredient, where the average size of the pharmaceutical granules is, for example, greater than 50 μm, greater than 100 μm, greater than 200 μm, greater than 400 μm, or greater than 600 μm, where granule size is determined by sieve analysis or by laser diffraction.

A pharmaceutical granulation can comprise, for example, an average granule size of less than 600 μm, less than 500 μm, less than 400 μm, less than 300 μm, or less than 200 μm, where granule size is determined by sieve analysis or by laser diffraction.

A pharmaceutical granulation can comprise a plurality of pharmaceutical granules having similar constituents and properties. For example, a pharmaceutical granulation can comprise and immediate-release granulation or can comprise a modified-release granulation.

A pharmaceutical granulation can comprise one or more populations of pharmaceutical granulations. Each of the two or more populations of pharmaceutical granulations can have a different composition and/or property, such as for example, average granule size, active pharmaceutical ingredient, release profile, and/or excipient composition.

Each of the two or more pharmaceutical granulations can have a different release profiles in the gastrointestinal tract.

A pharmaceutical granulation provided by the present disclosure can have an average granule size from for example, from 100 μm to 600 μm, from 200 μm to 600 μm, from 150 μm to 550, μm, from 200 μm to 500 μm, or from 250 μm to 450 μm, where the average granule size is determined by sieve analysis or laser diffraction.

A pharmaceutical granulation provided by the present disclosure can have an average a bulk density, for example, from 500 g/mL to 700 g/mL.

A pharmaceutical granulation provided by the present disclosure can have an average tapped density, for example, from 600 g/mL to 800 g/mL.

A pharmaceutical granulation provided by the present disclosure can have an average Hausner ratio, for example, from 1.1 to 1.25.

A pharmaceutical granulation provided by the present disclosure can have a friability, for example, from of less than 0.2 such as about 0.1 where friability is determined using an ionic sifter.

A pharmaceutical granulation can comprise an immediate-release pharmaceutical granulation, a modified-release pharmaceutical granulation, or a combination thereof.

A pharmaceutical composition can comprise one or more immediate-release pharmaceutical granulations and/or one or more modified-release pharmaceutical granulations.

A pharmaceutical granulation can comprise an immediate-release pharmaceutical granulation, a modified-release pharmaceutical granulation, or a combination thereof.

A pharmaceutical granulation can comprise an immediate release pharmaceutical granulation and a modified release pharmaceutical granulation.

An immediate-release pharmaceutical granulation comprises a plurality of immediate-release granules. Immediate-release granules refer to granules that completely disintegrate and/or dissolve when mixed with an aqueous solution at 25° C. in less than 90 seconds.

A modified-release pharmaceutical granulation comprises a plurality of modified-release granules.

In a pharmaceutical composition comprising an excipient granulation and a pharmaceutical granulation it can be desirable that the physical properties of the excipient granulation and the pharmaceutical granulation be similar. Having similar physical properties can minimize settling and/or maintain the integrity of the granulations during transport and storage. Relevant physical properties include, for example, average granule size, bulk density, tapped density, Hausner ration, compressibility index, Flodex value, and friability.

A pharmaceutical granulation can have an average granule size that is, for example, within ±20% of the average particle size of an excipient granulation with which the pharmaceutical granulation is combined, within ±15%, within ±10%, or within ±5% of the average particle size of an excipient granulation with which the pharmaceutical granulation is combined, where average granule size is determined by sieve analysis or laser diffraction.

A pharmaceutical granulation provided by the present disclosure can have an average bulk density that is, for example, within ±20% of the average bulk density of an excipient granulation with which the pharmaceutical granulation is combined, within ±15%, within ±10%, or within ±5% of the average bulk density of an excipient granulation with which the pharmaceutical granulation is combined, where average granule bulk density is determined using a bulk density cylinder.

A pharmaceutical granulation provided by the present disclosure can have an average bulk density, for example, from 0.6 g/mL to 0.9 g/mL, from 0.65 g/mL to 0.85 g/mL, or from 0.7 g/mL to 0.8 g/mL, where the average granule bulk density is determined using a bulk density cylinder.

A pharmaceutical granulation provided by the present disclosure can have an average tapped density that is, for example, within ±20% of the average tapped density of an excipient granulation with which the pharmaceutical granulation is combined, within ±15%, within ±10%, or within ±5% of the average tapped density of an excipient granulation with which the pharmaceutical granulation is combined, where average granule tapped density is determined according to USP <616>.

A pharmaceutical granulation provided by the present disclosure can have an average tapped density, for example, from 500 g/mL to 900 g/mL, from 550 g/L to 850 g/mL, from 600 g/mL to 800 g/mL, or from 650 g/mL to 750 g/mL, where the average granule tapped density is determined according to USP <616>.

A pharmaceutical granulation provided by the present disclosure can have a Hausner ratio that is, for example, within ±20% of the average Hausner ratio of an excipient granulation with which the pharmaceutical granulation is combined, within ±15%, within ±10%, or within ±5% of the Hausner ratio of an excipient with which the pharmaceutical granulation is combined, where the Hausner ratio is determined according to USP <1174>.

A pharmaceutical granulation provided by the present disclosure can have a Hausner ratio, for example, from 1.0 to 1.5, from 1.1 to 1.4, from 1.1 to 1.35, or from 1.1 to 1.25, where the Hausner ratio is determined according to USP <1174>.

A pharmaceutical granulation provided by the present disclosure can have a compressibility index that is, for example, within ±20% of the compressibility index of an excipient granulation with which the pharmaceutical granulation is combined, within ±15%, within ±10%, or within ±5% of the compressibility index of an excipient granulation with which the pharmaceutical granulation is combined.

A pharmaceutical granulation provided by the present disclosure can have a compressibility index, for example, from 8 to 22, from 10 to 20, or from 12 to 18.

A pharmaceutical granulation provided by the present disclosure can have a Flodex value that is, for example, within ±20% of the Flodex value of an excipient granulation with which the pharmaceutical granulation is combined, within ±15%, within ±10%, or within ±5% of the Flodex value of an excipient granulation with which the pharmaceutical granulation is combined, where the Flodex value is determined according to USP <1174>.

A pharmaceutical granulation provided by the present disclosure can have a Flodex value, for example, of less than 5 mm, less than 4 mm, less than 3 mm, or less than 2 mm, where the Flodex value is determined according to USP <1174>.

A pharmaceutical granulation provided by the present disclosure can have a friability value, for example, of less than 0.2 such as about 0.1 where friability is determined using an ionic sifter.

A pharmaceutical composition provided by the present disclosure can comprise an excipient granulation, one or more pharmaceutical granulations, and optionally one or more additional excipients.

A pharmaceutical composition provided by the present disclosure can comprise, for example, from 1 wt % to 15 wt % of an excipient granulation, from 3 wt % to 12 wt %, from 5 wt % to 10 wt %, or from 6 wt % to 9 wt % of an excipient granulation, where wt % is based on the total weight of the excipient granulation and the pharmaceutical granulation, or where wt % is based on the total weight of the pharmaceutical composition.

A pharmaceutical composition provided by the present disclosure can comprise, for example, from 85 wt % to 99 wt % of a pharmaceutical granulation, from 87 wt % to 97 wt %, or from 89 wt % to 95 wt %, of a pharmaceutical granulation, where wt % is based on the total weight of the excipient granulation and the pharmaceutical granulation, or where wt % is based on the total weight of the pharmaceutical composition.

A pharmaceutical composition provided by the present disclosure can comprise, for example, from 1 wt % to 15 wt % of an excipient granulation provided by the present disclosure; and from 85 wt % to 99 wt % of a pharmaceutical granulation, where wt % is based on the total weight of the excipient granulation and the pharmaceutical granulation, or where wt % is based on the total weight of the pharmaceutical composition.

A pharmaceutical composition provided by the present disclosure can comprise, for example, from 5 wt % to 10 wt % of an excipient granulation provided by the present disclosure; and from 90 wt % to 95 wt % of a pharmaceutical granulation, where wt % is based on the total weight of the excipient granulation and the pharmaceutical granulation, or wt % is based on the total weight of the pharmaceutical composition.

A pharmaceutical composition can further comprise one or more excipients that can be, for example, in powder form, and which are independent of the excipient granulation.

The one or more additional excipients can facilitate dispersion of an excipient granulation and a pharmaceutical granulation and/or minimize or prevent agglomeration of an excipient granulation and a pharmaceutical granulation.

The one or more additional excipients can comprise, for example, an antistatic agent.

Examples of suitable antistatic agents include talc (hydrous magnesium silicate), magnesium stearate, and silicon dioxide.

A pharmaceutical composition can comprise hydrous magnesium silicate (talc).

A pharmaceutical composition can comprise, for example, from 0.1 wt % to 1.0 wt %, from 0.2 wt % to 0.9 wt %, from 0.3 wt % to 0.8 wt %, or from 0.4 wt % to 0.7 wt % of an antistatic agent such as hydrous magnesium silicate, where wt % is based on the total weight of the pharmaceutical composition.

An oral pharmaceutical composition provided by the present disclosure can comprise a viscous aqueous solution and an active pharmaceutical ingredient. An oral pharmaceutical composition can be formed by combining an excipient granulation provided by the present disclosure and an active pharmaceutical ingredient with an aqueous solution such as water.

An active pharmaceutical ingredient can be dissolved in the viscous aqueous solution, can be a solid such as in the form of a pharmaceutical granulation, or can be a combination thereof.

An oral pharmaceutical composition provided by the present disclosure can comprise a pharmaceutical composition for oral ingestion prepared by mixing a pharmaceutical composition provided by the present disclosure with water or an aqueous solution to provide a viscous solution comprising an active pharmaceutical ingredient dissolved in the viscous solution and/or a pharmaceutical granulation comprising an active pharmaceutical ingredient dispersed and suspended in the viscous solution. The viscous solution comprises the constituents of the excipient granulation dissolved in water or the aqueous solution.

A product provided by the present disclosure can comprise an excipient granulation provided by the present disclosure contained within a package. The package can be any suitable package such as a container, a sachet, a vial, a bottle, a capsule, a pouch, a stick pack, a blister package, etc.

A pharmaceutical product provided by the present disclosure can comprise an excipient granulation provided by the present disclosure and an active pharmaceutical ingredient contained within a primary package.

A package can include any package suitable for use with pharmaceutical granulations.

Examples of suitable primary packages include blister packs, sachets, capsules, ampoules, pouches, stick packs, vials, etc.

In a pharmaceutical product, an active pharmaceutical ingredient can be in the form of liquid, a powder, a granulation, or other solid dosage from.

In a pharmaceutical product an active pharmaceutical ingredient can be in the form of a pharmaceutical granulation provided by the present disclosure.

In a pharmaceutical product an active pharmaceutical ingredient can be in the form of a pharmaceutical granulation provided by the present disclosure, where the pharmaceutical granulation comprises an immediate release pharmaceutical granulation and a modified release pharmaceutical granulation.

In a pharmaceutical product, the excipient granulation and the pharmaceutical granulation can be contained within the same primary package.

In a pharmaceutical product, the excipient granulation and the pharmaceutical granulation can be contained within separate primary packages.

In a pharmaceutical product comprising an immediate release pharmaceutical granulation and a modified release pharmaceutical granulation, the immediate release pharmaceutical granulation and a modified release pharmaceutical granulation can be contained within separate primary packages or within the same primary package.

In a pharmaceutical product, a package can contain a unit dose of the active pharmaceutical ingredient or less than a unit dose of the active pharmaceutical ingredient. In a pharmaceutical product comprising packages such as sachets comprising less than a unit dose of the active pharmaceutical ingredient, the pharmaceutical product can contain two or more packages containing the active pharmaceutical ingredient such that the total amount of the active pharmaceutical ingredient in the two or more packages makes up a unit dose of the active pharmaceutical ingredient.

A pharmaceutical product can comprise one or compartments containing an excipient granulation and the pharmaceutical granulation.

The excipient granulation and the pharmaceutical granulation can be contained within the same compartment of a package.

Each of the excipient granulation and the pharmaceutical granulation can be contained within separate, independent compartments of a common package. For example, a package can comprise a first compartment containing the excipient granulation and a second compartment containing the pharmaceutical granulation.

A pharmaceutical product can comprise a compartment containing the excipient granulation and a second compartment containing the pharmaceutical granulation where the compartments are configured to be mixed before or during combination with an aqueous solution.

A pharmaceutical product provided by the present disclosure can comprise a first package comprising an excipient granulation provided by the present disclosure and a second package comprising a pharmaceutical granulation.

A pharmaceutical product can comprise a sachet that contains both an excipient granulation provided by the present disclosure and a pharmaceutical granulation.

A pharmaceutical product can comprise, for example, from 0.5 g to 20 g of a combination of an excipient granulation provided by the present disclosure and a pharmaceutical granulation.

A pharmaceutical product provided by the present disclosure can comprise a unit dose of an active pharmaceutical ingredient.

A unit dose of the active pharmaceutical ingredient can be provided in one or more packages such as one or more sachets or one or more stick packs.

A pharmaceutical product provided by the present disclosure can comprise more than on unit dose of an active pharmaceutical ingredient. Each of the one or more unit doses can be provided in separate packages such as separate sachets or stick packs.

An oral pharmaceutical composition provided by the present disclosure can comprise a viscous aqueous solution. The oral pharmaceutical composition is intended for oral administration.

A viscous aqueous solution can be prepared by combining and mixing an excipient granulation provided by the present disclosure with an aqueous solution.

When excipient granulation is mixed with water, the contents of the excipient granulation provide a concentration, for example, from 0.005 g/mL to 0.08 g/mL from 0.005 g/mL to 0.060 g/mL of the excipient granules, from 0.010 g/mL to 0.055 g/mL, from 0.015 g/mL to 0.050 g/mL, from 0.015 g/mL to 0.045 g/mL, from 0.02 g/mL to 0.04 g/mL or from 0.025 g/mL to 0.035 g/mL of the excipient granulation, where g/mL is based on the grams of the excipient granulation mixed with the volume of water.

For example, an oral pharmaceutical composition can be prepared, for example, by mixing from 0.2 g to 2.2 g of the excipient granules in 40 mL of water, from 0.4 g to 2.0 g, from 0.6 g to 1.8 g, from 0.8 g to 1.6 g, from 1.0 g to 1.4 g of the excipient granules in 40 mL of water.

An oral pharmaceutical composition provided by the present disclosure can be prepared by combining an excipient granulation and a pharmaceutical granulation with water. The excipient granulation and the pharmaceutical granulation can be combined with water either simultaneously or sequentially. For example, an excipient granulation can first be combined with water to form a viscous solution, and a pharmaceutical granulation subsequently combined and mixed with the viscous solution. Alternatively, a pharmaceutical granulation can first be combined with water, and an excipient granulation subsequently added to the aqueous solution.

An oral pharmaceutical composition can comprise a viscous solution and an active pharmaceutical ingredient dissolved in the viscous solution and/or a pharmaceutical granulation comprising an active pharmaceutical ingredient suspended in the viscous solution.

An oral pharmaceutical composition can comprise an immediate release component and a modified release component. An immediate release component can comprise an active pharmaceutical ingredient dissolved in the viscous aqueous solution, an immediate release pharmaceutical granulation, or a combination thereof. A modified release component can comprise a modified release pharmaceutical granulation.

An immediate release component and a modified release component can comprise the same active pharmaceutical ingredient. An immediate release component and the modified release component can comprise a different active pharmaceutical ingredient.

An oral pharmaceutical composition can have a viscosity, for example, from 10 cP to 1,000 cP or from 20 cP to 800 cP at a temperature from 20° C. to 25° C. such as 23° C. An oral pharmaceutical composition can have a viscosity, for example, greater than 10 cP, greater than 20 cP, greater than 50 cP, greater than 100 cP, greater than 200 cP, greater than 400 cP, greater than 600 cP, or greater than 800 cP at a temperature from 20° C. to 25° C. such as 23° C. An oral pharmaceutical composition can have a viscosity, for example, of less than 1,000 cP, less than 800 cP, less than 600 cP, less than 400 cP, or less than 200 cP, at a temperature from 20° C. to 25° C. such as 23° C. Viscosity is determined using a Brookfield Digital Viscometer Model LVDV-I+ equipped with a Brookfield SC4-18 spindle at a spindle speed of 30 rpm. The oral pharmaceutical composition can be prepared by combining and mixing, for example, from 10 g to 20 g of a pharmaceutical composition with from 30 mL to 50 mL of water. The oral pharmaceutical composition can be prepared by combining and mixing, for example, from 12 g to 18 g of a pharmaceutical composition with from 35 mL to 45 mL of water. The oral pharmaceutical composition can have a concentration of the pharmaceutical composition, for example, from 0.20 g/mL to 0.70 g/mL, such as from 0.30 g/mL to 0.42 g/mL, or from 0.32 g/mL to 0.40 g/mL.

A pharmaceutical composition can comprise an excipient granulation and a pharmaceutical granulation comprising an immediate-release pharmaceutical granulation and a modified-release pharmaceutical granulation. When combined with a suitable amount of water, the excipient granulation can disintegrate to cause the viscosity of the solution to increase, the immediate release pharmaceutical granulation can dissolve to provide the active pharmaceutical ingredient dissolved in the viscous solution, and the modified-release pharmaceutical granulation can be suspended and dispersed in the viscous solution.

The pharmaceutical granulation can be configured to retain their integrity and dissolve and/or release the drug over time in the gastrointestinal track with a release profile determined by the constituents and the coatings of the modified-release pharmaceutical granules.

A method provided by the present disclosure can comprise combining an excipient granulation provided by the present disclosure with water to provide a viscous solution.

A method provided by the present disclosure can comprise a user opening a package comprising an excipient granulation provided by the present disclosure, pouring the excipient granulation into an aqueous solution, and mixing the excipient granules in the aqueous solution to provide a viscous solution.

A method provided by the present disclosure can comprise a user opening a package comprising an excipient granulation provided by the present disclosure and an active pharmaceutical ingredient, pouring the excipient granulation and the active pharmaceutical ingredient into an aqueous solution, and mixing the excipient granulation and the pharmaceutical granulation in the aqueous solution to provide a viscous aqueous solution containing the active pharmaceutical ingredient. The active pharmaceutical ingredient can dissolve in the viscous aqueous solution or can be contained in a pharmaceutical granulation or other solid dosage form that is suspended in the viscous aqueous solution.

When combined with a suitable amount of water a pharmaceutical composition provided by the present disclosure is configured to provide a solution having a viscosity capable of suspending a pharmaceutical granulation. The suspension can comprise pharmaceutical granules suspended in the viscous solution.

The aqueous solution can comprise, for example, from 25 mL to 250 mL, from 25 mL to 200 mL, from 25 mL to 150 mL, from 25 mL to 100 mL, or from 25 mL to 75 mL.

The aqueous solution can be water.

Pharmaceutical compositions provided by the present disclosure can be included in a kit that can be used to administer the compound to a patient for therapeutic purposes. A kit can include a pharmaceutical composition suitable for administration to a patient and instructions for administering the pharmaceutical composition to the patient. The kit can comprise a pharmaceutical composition provided by the present disclosure and instructions for combining the contents of the kit with an aqueous solution to provide an oral pharmaceutical composition and with instructions or administering the oral pharmaceutical composition to a patient.

A kit can comprises a primary package containing a pharmaceutical composition and a secondary package.

A pharmaceutical composition provided by the present disclosure can be included in a container, package, or dispenser together with instructions for administration.

Instructions supplied with a kit may be printed and/or supplied, for example, as an electronic-readable medium, a video cassette, an audiotape, a flash memory device, or may be published on an internet web site or distributed to a patient and/or health care provider as an electronic communication.

A pharmaceutical composition provided by the present disclosure can be used to treat a disease in a patient.

Methods of treating a disease in a patient comprise orally administering to a patient in need of such treatment an oral pharmaceutical composition prepared from a pharmaceutical composition provided by the present disclosure, wherein the disease is capable of being treated by active pharmaceutical ingredient; and wherein the pharmaceutical composition comprises a therapeutically effective amount of the active pharmaceutical ingredient for treating the disease. The pharmaceutical composition can comprise a pharmaceutical granulation.

An excipient granulation provided by the present disclosure can be used in combination with an active pharmaceutical ingredient in the manufacture of a medicament.

A pharmaceutical composition provided by the present disclosure can be used in the manufacture of a medicament. The medicament can comprise, for example, an active pharmaceutical ingredient dissolved in a viscous solution and/or a pharmaceutical granulation suspended in a viscous solution.

An excipient granulation provided by the present disclosure in combination with an active pharmaceutical ingredient can be used to prepare an oral pharmaceutical composition.

A pharmaceutical composition provided by the present disclosure can be used to prepare an oral pharmaceutical composition.

Methods provided by the present disclosure include combining an excipient granulation provided by the present disclosure with an aqueous solution to increase the viscosity of the aqueous solution. Methods provided by the present disclosure include combining an excipient granulation provided by the present disclosure to enhance the palatability of an oral pharmaceutical composition, such as an oral pharmaceutical composition comprising a pharmaceutical granulation.

### ASPECTS OF THE INVENTION

The invention is further defined by one or more of the following aspects.

Aspect 1. An excipient granulation, wherein the excipient granulation comprises a plurality of excipient granules, wherein the excipient granules comprise: a viscosifying agent; a disintegrant; and one or more additional excipients.

Aspect 2. The excipient granulation of aspect 1, wherein the excipient granules comprise: from 1 wt % to 35 wt % of the viscosifying agent; from 1 wt % to 25 wt % of the disintegrant; and from 40 wt % to 98 wt % of the one or more additional excipients; wherein wt % is based on the total weight of the excipient granules.

Aspect 3. The excipient granulation of aspect 1, wherein the excipient granules comprise: from 5 wt % to 15 wt % of the viscosifying agent; from 5 wt % to 20 wt % of the disintegrant; and from 65 wt % to 90 wt % of the one or more additional excipients; wherein wt % is based on the total weight of the excipient granules.

Aspect 4. The excipient granulation of any one of aspects 1 to 3, wherein the excipient granules have a homogeneous composition.

Aspect 5. The excipient granulation of any one of aspects 1 to 4, wherein the excipient granules have an average particle size from 100 μm to 600 μm, where the particle size is determined by sieve analysis or laser diffraction.

Aspect 6. The excipient granulation of any one of aspects 1 to 5, wherein the excipient granules have an average bulk density from 0.5 g/mL to 0.7 g/mL, wherein the bulk density is determined according to USP <616>, Method 1.

Aspect 7. The excipient granulation of any one of aspects 1 to 6, wherein the viscosifying agent comprises xanthan gum.

Aspect 8. The excipient granulation of aspect 7, wherein the xanthan gum has an average particle size from 25 μm to 100 μm, wherein the particle size is determined using sieve analysis or laser diffraction.

Aspect 9. The excipient granulation of aspect 7, wherein the xanthan gum has an average particle size of less than 75 μm, wherein the particle size is determined using sieve analysis or laser diffraction.

Aspect 10. The excipient granulation of any one of aspects 7 to 9, wherein the xanthan gum has a viscosity ratio from 1.0 to 1.5.

Aspect 11. The excipient granulation of any one of aspects 7 to 10, wherein the xanthan gum provides a viscosity from 1,200 mPa×sec to 1600 mPa×sec (cP) determined using a Brookfield digital viscometer Model LVDV-1+ equipped with a Brookfield SC4-18 spindle at a spindle speed of 30 RPM.

Aspect 12. The excipient granulation of any one of aspects 1 to 11, wherein the disintegrant comprises crospovidone.

Aspect 13. The excipient granulation of aspect 12, wherein the crospovidone has: a pH value from 5 to 8 as a 1% solution in water at 25° C.; a bulk density from 0.25 g/cm³ to 0.45 g/cm³′ and/or an average particle size from 20 μm to 40 μm.

Aspect 14. The excipient granulation of any one of aspects 1 to 13, wherein the one or more additional excipients comprises a diluent, a binder, a flavoring agent, a buffer, an acid, a sweetening agent, a filler, or a combination of any of the foregoing.

Aspect 15. The excipient granulation of any one of aspects 1 to 14, wherein the one or more additional excipients comprises a binder.

Aspect 16. The excipient granulation of aspect 15, wherein the binder comprises polyvinylpyrrolidone.

Aspect 17. The excipient granulation of aspect 16, wherein the polyvinylpyrrolidone has: a K-value from 29 to 32; a number average molecular weight of 58,000 Daltons; a viscosity of 2.5 cP in a 5% aqueous solution at 25° C.; an average particle size of 80 μm; a bulk density of 0.36 g/cm³; and/or a tapped density of 0.47 g/cm³.

Aspect 18. The excipient granulation of any one of aspects 14 to 17, wherein, the excipient granules comprise from 5 wt % to 20 wt % of the binder; and wt % is based on the total weight of the excipient granules.

Aspect 19. The excipient granulation of any one of aspects 1 to 18, wherein the one or more additional excipients comprises a diluent.

Aspect 20. The excipient granulation of aspect 19, wherein the diluent comprises anhydrous dibasic calcium phosphate.

Aspect 21. The excipient granulation of any one of aspects 19 to 20, wherein, the excipient granules comprise from 10 wt % to 30 wt % of the diluent; and wt % is based on the total weight of the excipient granules.

Aspect 22. The excipient granulation of any one of aspects 1 to 21, wherein the one or more additional excipients comprises a filler.

Aspect 23. The excipient granulation of aspect 22, wherein the filler comprises microcrystalline cellulose.

Aspect 24. The excipient granulation of any one of aspects 22 to 23, wherein, the excipient granules comprise from 1 wt % to 20 wt % of the binder; and wt % is based on the total weight of the excipient granules.

Aspect 25. The excipient granulation of any one of aspects 1 to 24, wherein the one or more additional excipients comprises a flavoring agent.

Aspect 26. The excipient granulation of aspect 25, wherein the flavoring agent comprises a citric fruit flavoring agent, a berry flavoring agent, a punch flavoring agent, or a combination of any of the foregoing.

Aspect 27. The excipient granulation of any one of aspects 25 to 26, wherein, the excipient granules comprise from 15 wt % to 35 wt % of the flavoring agent; and wt % is based on the total weight of the excipient granules.

Aspect 28. The excipient granulation of any one of aspects 1 to 27, wherein the one or more additional excipients comprises a buffer.

Aspect 29. The excipient granulation of aspect 28, wherein the buffer comprises sodium citrate anhydrate.

Aspect 30. The excipient granulation of any one of aspects 28 to 29, wherein, the excipient granules comprise from 0.5 wt % to 6.5 wt % of the buffer; and wt % is based on the total weight of the excipient granules.

Aspect 31. The excipient granulation of any one of aspects 1 to 30, wherein the one or more additional excipients comprises an acid.

Aspect 32. The excipient granulation of aspect 31, wherein the acid comprises citric acid anhydrous.

Aspect 33. The excipient granulation of any one of aspects 31 to 32, wherein, the excipient granules comprise from 0.5 wt % to 8 wt % of the acid; and wt % is based on the total weight of the excipient granules.

Aspect 34. The excipient granulation of any one of aspects 1 to 33, wherein the one or more additional excipients comprises a sweetening agent.

Aspect 35. The excipient granulation of aspect 34, wherein the sweetening agent comprises sucralose.

Aspect 36. The excipient granulation of any one of aspects 34 to 35, wherein, the excipient granules comprise from 0.1 wt % to 6 wt % of the sweetening agent; and wt % is based on the total weight of the excipient granules.

Aspect 37. The excipient granulation of aspect 14, wherein the excipient granules comprise: from 2 wt % to 22 wt % of the binder; from 10 wt % to 30 wt % of the diluent; from 1 wt % to 20 wt % of the filler; from 15 wt % to 35 wt % of the flavoring agent; from 0.5 wt % to 6.5 wt % of the buffer; from 0.5 wt % to 8 wt % of the acid; and/or from 0.1 wt % to 6 wt % of the sweetening agent, wherein wt % is based on the total weight of the excipient granules.

Aspect 38. The excipient granulation of aspect 37, wherein the excipient granules comprise: from 1 wt % to 35 wt % of the viscosifying agent; and from 1 wt % to 25 wt % of the disintegrant; wherein wt % is based on the total weight of the excipient granules.

Aspect 39. The excipient granulation of aspect 14, wherein the excipient granules comprise: from 5 wt % to 15 wt % of the binder; from 17 wt % to 27 wt % of the diluent; from 6 wt % to 16 wt % of the filler; from 20 wt % to 30 wt % of the flavoring agent; from 1 wt % to 5 wt % of the buffer; from 2 wt % to 6 wt % of the acid; and/or from 1 wt % to 5 wt % of the sweetening agent, wherein wt % is based on the total weight of the excipient granules.

Aspect 40. The excipient granulation of aspect 39, wherein the excipient granules comprise: from 5 wt % to 15 wt % of the viscosifying agent; and from 7 wt % to 17 wt % of the disintegrant; wt % is based on the total weight of the excipient granules.

Aspect 41. The excipient granulation of any one of aspects 14 to 40, wherein, the viscosifying agent comprises xanthan gum; the disintegrant comprises crospovidone; the binder comprises polyvinylpyrrolidone; the diluent comprises dicalcium phosphate; the filler comprises microcrystalline cellulose; the flavoring agent comprises a berry/punch flavoring agent; the buffer comprises sodium citrate dihydrate; the acid comprises citric acid anhydrous; and the sweetening agent comprises sucralose, wherein wt % is based on the total weight of the excipient granules.

Aspect 42. The excipient granulation of any one of aspects 1 to 41, wherein, the excipient granules have an average particle size from 200 μm to 500 μm; and the particle size is determined by sieve analysis or laser diffraction.

Aspect 43. The excipient granulation of any one of aspects 1 to 39, wherein, the excipient granules have an average particle size from 250 μm to 400 μm; and the particle size is determined by sieve analysis or laser diffraction.

Aspect 44. The excipient granulation of any one of aspects 1 to 39, wherein, the excipient granules have a particle size distribution characterized by 8.5 wt % greater than 35 mesh (500 μm), 39 wt % from 35-60 mesh (500-250 μm), 52.2% pan, less than 35 mesh (500 μm) 52.2 wt %; and the particle size is determined by sieve analysis or laser diffraction.

Aspect 45. The excipient granulation of any one of aspects 1 to 44, wherein, the excipient granulation (35-60 mesh fraction) has an average bulk density from 0.5 g/mL to 0.9 g/mL; and the bulk density is determined using a bulk density cylinder.

Aspect 46. The excipient granulation of any one of aspects 1 to 44, wherein, the excipient granulation has an average bulk density from 0.6 g/mL to 0.7 g/mL (35-60 mesh fraction); and the bulk density is determined using a bulk density cylinder.

Aspect 47. The excipient granulation of any one of aspects 1 to 46, wherein the excipient granulation, when reconstituted in water at 25° C., completely dissolves in less than 60 seconds.

Aspect 48. The excipient granulation of any one of aspects 1 to 47, wherein the excipient granulation, when reconstituted in in water at 25° C., completely dissolves in less than 30 seconds.

Aspect 49. The excipient granulation of any one of aspects 1 to 47, wherein the excipient granulation, when reconstituted as a 1 w/v % solution in water at 25° C., completely dissolves in less than 60 seconds.

Aspect 50. The excipient granulation of any one of aspects 1 to 47, wherein the excipient granulation, when reconstituted as a 1 w/v % solution in water at 25° C., completely dissolves in less than 30 seconds.

Aspect 51. The excipient granulation of any one of aspects 1 to 50, wherein the excipient granulation has an average Hausner ratio from 1.1 to 1.25, wherein the Hausner ratio is determined according to USP <1174>.

Aspect 52. The excipient granulation of any one of aspects 1 to 51, wherein the excipient granulation has an average compressibility index from 12 to 18.

Aspect 53. The excipient granulation of any one of aspects 1 to 52, wherein the excipient granulation has an average Flodex value of less than 5 mm.

Aspect 54. The excipient granulation of any one of aspects 1 to 53, wherein, the excipient granulation has an average friability of less than 2 wt %; and the friability is determined using a sonic sifter.

Aspect 55. The excipient granulation of any one of aspects 1 to 54, wherein the excipient granulation provides a pH from 4.0 to 8.0 when reconstituted as a 1 w/v % solution of water.

Aspect 56. The excipient granulation of any one of aspects 1 to 55, wherein, when 1.2 g of the excipient granulation is reconstituted in 40 mL of water at 23° C., the resulting solution exhibits a viscosity vs. time profile as shown in FIG. 2.

Aspect 57. The excipient granulation of any one of aspects 1 to 52, wherein, when 1.2 g of the excipient granulation is reconstituted in 40 mL of water at 23° C., the resulting solution exhibits a luminosity vs. time profile as shown in FIG. 3.

Aspect 58. The excipient granulation of any one of aspects 1 to 57, wherein after mixing a solution of 1.2 g of the excipient granulation and 40 mL of water at 23° C. for 90 seconds, the solution has a viscosity within a range from 45 cP to 80 cP, where viscosity is determined using a Brookfield Digital Viscometer Model LVDV-I+ equipped with a Brookfield SC4-18 spindle at a spindle speed of 30 rpm.

Aspect 59. The excipient granulation of any one of aspects 1 to 57, wherein after mixing a solution of 1.2 g of the excipient granulation and 40 mL of water at 23° C. for 30 seconds, the solution has a viscosity within a range from 15 cP to 40 cP, where viscosity is determined using a Brookfield Digital Viscometer Model LVDV-I+ equipped with a Brookfield SC4-18 spindle at a spindle speed of 30 rpm.

Aspect 60. The excipient granulation of any one of aspects 1 to 57, wherein after mixing a solution of 1.2 g of the excipient granulation and 40 mL of water at 23° C. for 60 seconds, the solution has a viscosity within a range from 35 cP to 60 cP, where viscosity is determined using a Brookfield Digital Viscometer Model LVDV-I+ equipped with a Brookfield SC4-18 spindle at a spindle speed of 30 rpm.

Aspect 61. The excipient granulation of any one of aspects 1 to 60, wherein the excipient granules are made using wet granulation.

Aspect 62. A method of making the excipient granules of any one of aspects 1 to 61 comprising: dry mixing the viscosifying agent, the disintegrant, and the one or more excipients to provide a dry mixture; granulating the dry mixture with a buffer solution to provide a first granulation; wet massing the first granulation to form a first wet mass; granulating the first wet mass with water to provide a second granulation; wet massing the second granulation to form a second wet mass; and drying the second wet mass to provide the excipient granules.

Aspect 63. An excipient granulation comprising the excipient granules prepared using the method of aspect 62.

Aspect 64. A composition comprising the excipient granulation of any one of aspects 1 to 61 and 63.

Aspect 65. The composition of aspect 64, wherein the composition comprises: from 1 wt % to 15 wt % of the excipient granulation; and wherein wt % is based on the total weight of the composition.

Aspect 66. A pharmaceutical composition comprising: the excipient granulation of any one of aspects 1 to 61 and 63; and an active pharmaceutical ingredient.

Aspect 67. The pharmaceutical composition of aspect 65, wherein the active pharmaceutical ingredient is in the form of a solid dosage form.

Aspect 68. The pharmaceutical composition of aspect 67, wherein the solid dosage form is in the form of a powder, a granulation, or a tablet.

Aspect 69. The pharmaceutical composition of any one of aspects 66 to 68, wherein the pharmaceutical composition comprises: from 1 wt % to 15 wt % of the excipient granulation; and wherein wt % is based on the total weight of the pharmaceutical composition.

Aspect 70. The pharmaceutical composition of any one of aspects 66 to 69, wherein the pharmaceutical composition comprises a pharmaceutical granulation, wherein the pharmaceutical granulation comprises the active pharmaceutical ingredient.

Aspect 71. The pharmaceutical composition of aspect 70, wherein the pharmaceutical granulation comprises a plurality of immediate release pharmaceutical granules, a plurality of modified release pharmaceutical granules, or a combination thereof.

Aspect 72. The pharmaceutical composition of any one of aspects 70 to 71, wherein the pharmaceutical composition comprises: from 1 wt % to 15 wt % of the excipient granulation; and from 85 wt % to 99 wt % of the pharmaceutical granulation, wherein wt % is based on the total weight of the pharmaceutical composition.

Aspect 73. The pharmaceutical composition of any one of aspects 70 to 72, wherein the pharmaceutical composition comprises: from 5 wt % to 11 wt % of the excipient granulation; and from 89 wt % to 95 wt % of the pharmaceutical granulation, wherein wt % is based on the total weight of the pharmaceutical composition.

Aspect 74. The pharmaceutical composition of any one of aspects 64 to 73, wherein the pharmaceutical composition comprises an antistatic agent.

Aspect 75. The pharmaceutical composition of aspect 74, wherein the antistatic agent comprises hydrous magnesium silicate.

Aspect 76. The pharmaceutical composition of any one of aspects 74 to 75, wherein the pharmaceutical composition comprises from 0.1 wt % to 1.0 wt % of the antistatic agent, wherein wt % is based on the total weight of the pharmaceutical composition.

Aspect 77. The pharmaceutical composition of any one of aspects 70 to 76, wherein, the excipient granulation has a first bulk density; the pharmaceutical granulation has a second bulk density; and the first bulk density is within ±20% of the second bulk density.

Aspect 78. The pharmaceutical composition of any one of aspects 70 to 77, wherein, the excipient granulation has a first average particle size; the pharmaceutical granulation has a second average particle size; and the first average particle size is within ±20% of the second average particle size.

Aspect 79. The pharmaceutical composition of any one of aspects 70 to 78, wherein, the excipient granulation has a first tapped density; the pharmaceutical granulation has a second tapped; and the first average tapped density is within ±20% of the tapped density.

Aspect 80. The pharmaceutical composition of any one of aspects 70 to 79, wherein, the excipient granulation has a first Hausner ratio; the pharmaceutical granulation has a second Hausner ratio; and the first Hausner ratio is within ±20% of the second Hausner ratio.

Aspect 81. The pharmaceutical composition of any one of aspects 70 to 80, wherein, the excipient granulation has a first compressibility index; the pharmaceutical granulation has a second compressibility index; and the first compressibility index is within ±20% of the second compressibility index.

Aspect 82. The pharmaceutical composition of any one of aspects 70 to 81, wherein, the excipient granulation has a first Flodex value; the pharmaceutical granulation has a second Flodex value; and the first Flodex value is within ±20% of the second Flodex value.

Aspect 83. The pharmaceutical composition of any one of aspects 70 to 82, wherein, the excipient granulation has a first friability value; the pharmaceutical granulation has a second friability value; and the first friability value is within ±20% of the second friability value.

Aspect 84. A product comprising the excipient granulation of any one of aspects 1 to 61 and 63 or the composition of any one of aspects 64 to 65.

Aspect 85. A pharmaceutical product comprising the pharmaceutical composition of any one of aspects 66 to 69.

Aspect 86. A pharmaceutical product comprising the pharmaceutical composition of any one of aspects 70 to 83.

Aspect 87. The pharmaceutical product of aspect 86, wherein the product comprises the excipient granulation, the pharmaceutical granulation, or a combination thereof contained within a package.

Aspect 88. The pharmaceutical product of aspect 87, wherein the package comprises a sachet.

Aspect 89. The pharmaceutical product of any one of aspects 85 to 88, wherein the pharmaceutical product comprises the excipient granulation and a pharmaceutical granulation combined in the same package.

Aspect 90. The pharmaceutical product of any one of aspects 85 to 88, wherein the pharmaceutical product comprises a first package containing the excipient granulation and a second package containing the pharmaceutical granulation.

Aspect 91. The pharmaceutical product of any one of aspects 85 to 88, wherein the pharmaceutical product comprises a package, wherein the package comprises a first compartment containing the excipient granulation and a second compartment containing the pharmaceutical granulation.

Aspect 92. The pharmaceutical product of aspect 91, wherein the package is configured to combine contents of the first compartment with contents of the second compartment during use to provide a pharmaceutical composition.

Aspect 93. The pharmaceutical product of any one of aspects 85 to 92, wherein the pharmaceutical product comprises from 0.5 g to 20 g of the pharmaceutical composition.

Aspect 94. A composition for oral administration prepared using the excipient granulation of any one of aspects 1 to 61 and 63.

Aspect 95. An oral pharmaceutical composition for oral administration prepared using the excipient granulation of any one of aspects 66 to 83.

Aspect 96. The oral pharmaceutical composition of aspect 95, wherein the oral pharmaceutical composition comprises water.

Aspect 97. The oral pharmaceutical composition of any one of aspects 95 to 96, wherein the oral pharmaceutical composition has a viscosity from 50 cP to 1,000 cP, wherein the viscosity is determined using a Brookfield Digital Viscometer Model LVDV-I+ equipped with a Brookfield SC4-18 spindle at a spindle speed of 30 rpm at a temperature of 23° C.

Aspect 98. The oral pharmaceutical composition of any one of aspects 95 to 97, wherein the oral pharmaceutical composition comprises an immediate release component, a modified release component, or a combination thereof.

Aspect 99. The oral pharmaceutical composition of any one of aspects 95 to 98, wherein the oral pharmaceutical composition comprises an immediate release pharmaceutical granulation, a modified release pharmaceutical granulation, or a combination thereof.

Aspect 100. The oral composition of aspect 98, wherein the immediate release component comprises an active pharmaceutical ingredient dissolved in a viscous aqueous solution.

Aspect 101. The oral pharmaceutical composition of any one of aspects 95 to 100, wherein the oral pharmaceutical composition comprises a pharmaceutical granulation suspended in a viscous aqueous solution.

Aspect 102. The oral pharmaceutical composition of any one of aspects 95 to 101, wherein the oral pharmaceutical composition comprises a modified release component suspended in a viscous aqueous solution.

Aspect 103. The oral pharmaceutical composition of aspect 98, wherein the immediate release component comprises an immediate release pharmaceutical granulation.

Aspect 104. The oral pharmaceutical composition of any one of aspects 98 to 103, wherein the modified release component comprises a modified release pharmaceutical granulation.

Aspect 105. The oral pharmaceutical composition of aspect 94, wherein the modified release granulation is suspended in the viscous aqueous solution.

Aspect 106. The oral pharmaceutical composition of aspect 95, wherein the viscous aqueous solution has a viscosity of from 50 cP to 600 cP at a temperature from 20° C. to 25° C. such as 23° C., wherein the viscosity is determined using a Brookfield digital viscometer Model LVDV-1+ equipped with a Brookfield SC4-18 spindle at a spindle speed of 30 RPM.

Aspect 107. A kit comprising the excipient granulation of any one of aspects 1 to 61 and 63, the composition of any one of aspects 64 to 65, the pharmaceutical composition of any one of aspects 66 to 83, the product of aspect 84, the pharmaceutical product of any one of aspects 85 to 93, or the oral pharmaceutical composition of any one of aspects 95 to 106, and instructions for using contents of the kit.

Aspect 108. A method of using the pharmaceutical product of any one of aspects 85 to 93 comprising: opening the pharmaceutical package; pouring the pharmaceutical composition into water; and mixing the contents to provide an oral pharmaceutical composition.

Aspect 109. The method of aspect 108, wherein the water comprises from 10 mL to 60 mL of water.

Aspect 110. The method of any one of aspects 108 to 109, wherein the pharmaceutical composition comprises from 1 g to 3 g of the excipient granules.

Aspect 111. The method of any one of aspects 108 to 110, wherein the method further comprises, before combining the contents with water, combining and mixing the excipient granulation and the drug-containing granulation to form a pharmaceutical composition.

Aspect 112. The method of any one of aspects 108 to 111, wherein the method comprises, after pouring the excipient granulation into water to form a viscous solution, combining the drug-containing granulation with the viscous solution to provide a pharmaceutical composition.

Aspect 113. A method of treating a disease in a patient comprising orally administering to a patient in need of such treatment an oral pharmaceutical composition prepared from the pharmaceutical composition of any one of aspects 66 to 83, wherein the disease is capable of being treated by the drug; and wherein the pharmaceutical composition comprises a therapeutically effective amount of the drug for treating the disease.

Aspect 114. A method of treating a disease in a patient comprising orally administering to a patient in need of such treatment the oral pharmaceutical composition of any one of aspects 95 to 106, wherein the disease is capable of being treated by the active pharmaceutical ingredient; and wherein the pharmaceutical composition comprises a therapeutically effective amount of the active pharmaceutical ingredient for treating the disease.

Aspect 115. The method of any one of aspects 113 to 114, wherein pharmaceutical composition comprises a pharmaceutical granulation.

Aspect 116. Use of the excipient granulation of any one of aspects 1 to 61 in the manufacture of a medicament.

Aspect 117. Use of the pharmaceutical composition of any one of aspects 66 to 83 in the manufacture of a medicament.

Aspect 118. The use of any one of aspects 116 to 117, wherein the medicament comprises an oral pharmaceutical composition.

Aspect 119. The use of any one of aspects 116 to 118, wherein the medicament comprises a pharmaceutical granulation suspended in a viscous aqueous solution.

Aspect 120. A method of using the excipient granules of any one of aspects 1 to 61 to increase the viscosity of an aqueous solution.

Aspect 121. A method of using the excipient granules of any one of aspects 1 to 61 to enhance the palatability of an oral pharmaceutical composition.

### EXAMPLES

Embodiments provided by the present disclosure are further illustrated by reference to the following examples, which describe excipient granulations, properties of excipient granulations, and compositions comprising excipient granulations. It will be apparent to those skilled in the art that many modifications, both to materials, and methods, may be practiced without departing from the scope of the disclosure. It should be noted that there are alternative ways of implementing the embodiments disclosed herein. Accordingly, the present embodiments are to be considered as illustrative and not restrictive. Furthermore, the claims are not to be limited to the details given herein and are entitled their full scope and equivalents thereof.

### Example 1

### Excipient Granulations

An excipient granulation was prepared using high-shear dry granulation and high shear wet granulation.

Formulation development work with various excipients was undertaken to identify excipient granule formulations that rapidly formed a viscous solution after being reconstituted with 40 mL of water and were suitable for oral administration.

The methods to produce excipient granules were (1) dry granulation through roller compaction and/or slugging on a tablet press, and (2) high-shear wet granulation using either a small coffee grinder for small scale experiments or on a 4 L bowl high-shear granulator for the larger batch size.

An example of an excipient granule composition is provided in Table 1.

TABLE 1
Example of excipient granule constituents.

| Component | Function | % w/w | mg/mL |
|---|---|---|---|
| Xanthan Gum | Viscosifying | 10.0 | 3.0 |
| | Agent | | |
| Dicalcium Phosphate | Binder | 21.5 | 6.5 |
| Microcrystalline | Filler | 10.8 | 3.2 |
| cellulose | | | |
| Flavoring Agent | Flavoring Agent | 25.0 | 7.5 |
| Crospovidone | Disintegrant | 12.0 | 3.6 |
| Polyvinylpyrrolidone | Water-soluble | 10.0 | 3.0 |
| | Polymer | | |
| Sodium Citrate | Buffer | 3.3 | 1.0 |
| Dihydrate | | | |
| Citric Acid Anhydrous | Acid | 4.2 | 1.3 |
| Sucralose | Sweetening Agent | 3.2 | 1.0 |
| Total | — | 100.0 | 30.0 |

Excipient granules were prepared by dry mixing the constituents and then granulating the dry mixture with a buffer comprising the sodium citrate dihydrate and the citric acid anhydrous dissolved in water to provide a first granulation. The first granulation was wet massed to provide a first wet mass, which was then granulated with water to provide a second granulation. The second granulation was wet massed to provide a second wet mass and dried to provide the excipient granules.

A Collette (GEA) GRAL 25 high shear granulator equipped with a 25 L jacked bowl was used for granulation. The jacketed bowl was coupled to an Advantage Engineering Air cooled water chiller set at 46° F. (8° C.). Buffer or water was sprayed into the mixture using a two fluid nozzle with a 1-mm nozzle orifice at an atomization pressure of 45 bar.

The excipient granules were dried using a Freund-Vector FLM-3 fluid bed drier at 60° C.

The dried excipient granules were milled using a Quadro Comil® 197 equipped with 50 G or 32 G screens and sieved using a tabletop vibratory sieving base equipped with #35 and #70 mesh screens. Fines (<210 μm) passing through the #70 mesh screen were removed.

### Example 2

### Properties of Excipient Granulation

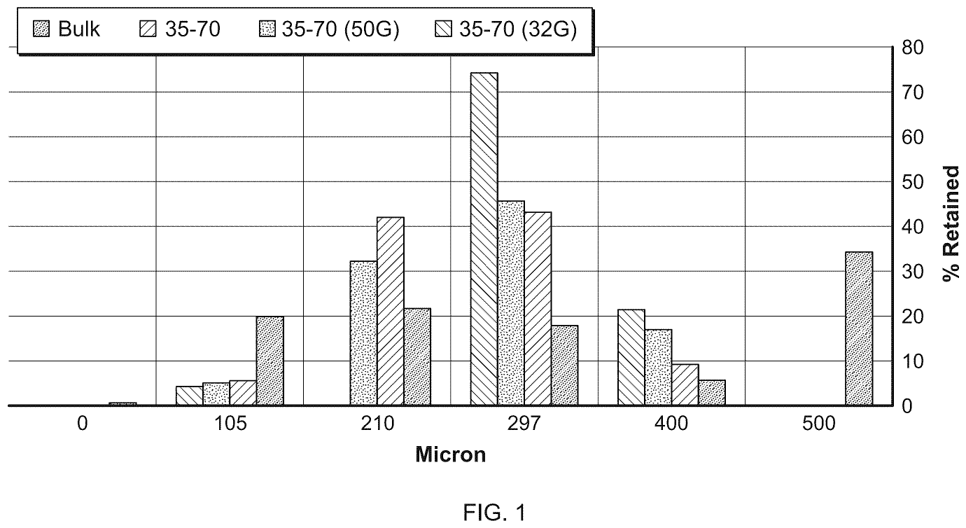

FIG. 1 shows the particle size distribution of the excipient granules prepared in Example 1 at different stages in the sieving process. The size distributions include the distributions of the bulk granules before sieving (bulk), the 35 G-75 G screen fraction (35-70), after passing through the 50 G screen (50 G), after passing through the 32 G screen (30 G). The particle size distribution was determined using sieve analysis or laser diffraction.

The excipient granules (1.2 g) were combined and mixed with 40 mL of water to provide a reconstituted viscous solution. The pH of the reconstituted viscous solution was 4.3.

FIG. 2 shows the reconstitution viscosity profile for various excipient granulations comprising different constituents and prepared using different granulation and wet massing conditions.

The reconstitution viscosity profile for the excipient granules of Example 1 is shown in FIG. 2 as the thick line(O). Other examples are shown as lines A-N.

The reconstitution viscosity was determined by combining 1.2 g or the excipient granules in 40 mL of water at a temperature of 23° C. and mixing using a Brookfield Digital Viscometer Model LVDV-I+ equipped with a Brookfield SC4-18 spindle at a spindle speed of 30 rpm.

The reconstitution luminosity profile for the excipient granulation of Example 1 and other excipient granules is shown in FIG. 3 as the thick line (O). Other examples are shown as lines A-N.

Luminosity was measured by combining and mixing the excipient granules of Example 1 with 40 mL of water at a temperature of 23° C. and determining the viscosity using a Technical Color Solutions Datacolor™ 650 spectrophotometer.

The excipient granules of Example 1 had a bulk density from 0.58 g/mL to 0.62 g/mL, a tapped density from 0.69 g/mL to 0.74 g/mL, a Hausner Ratio from 0.18 to 1.20, a compressibility index from 15.5 to 16.8, and a Flodex value of 4.

It should be noted that there are alternative ways of implementing the embodiments disclosed herein. Accordingly, the present embodiments are to be considered as illustrative and not restrictive. Furthermore, the claims are not to be limited to the details given herein and are entitled their full scope and equivalents thereof.

What is claimed is:

1. An excipient granulation comprising a plurality of excipient granules, wherein the excipient granules comprise:
a viscosifying agent;
a disintegrant; and
one or more additional excipients.

2. The excipient granulation of claim 1, wherein the excipient granules comprise:
from 1 wt % to 35 wt % of the viscosifying agent;
from 1 wt % to 25 wt % of the disintegrant; and
from 40 wt % to 98 wt % of the one or more additional excipients;
wherein wt % is based on the total weight of the excipient granules.

3. The excipient granulation of claim 1, wherein the excipient granules have an average particle size from 100 μm to 600 μm, where the particle size is determined by sieve analysis or laser diffraction.

4. The excipient granulation of claim 1, wherein the viscosifying agent comprises xanthan gum.

5. The excipient granulation of claim 1, wherein the disintegrant comprises crospovidone.

6. The excipient granulation of claim 1, wherein the one or more additional excipients comprises a diluent, a binder, a flavoring agent, a buffer, an acid, a sweetening agent, a filler, or a combination of any of the foregoing.

7. The excipient granulation of claim 1, wherein after mixing a solution of 1.2 g of the excipient granulation and 40 mL of water at a temperature of 23° C. for 90 seconds, the solution has a viscosity within a range from 40 cP to 100 cP, wherein the viscosity is determined using a Brookfield digital viscometer Model LVDV-1+ equipped with a Brookfield SC4-18 spindle at a spindle speed of 30 RPM.

8. A pharmaceutical composition comprising:
the excipient granulation of claim 1; and
an active pharmaceutical ingredient.

9. The pharmaceutical composition of claim 8, wherein the pharmaceutical composition comprises:
from 1 wt % to 15 wt % of the excipient granulation; and
wherein wt % is based on the total weight of the pharmaceutical composition.

10. The pharmaceutical composition of claim 8, wherein the pharmaceutical composition comprises a pharmaceutical granulation, wherein the pharmaceutical granulation comprises the active pharmaceutical ingredient.

11. The pharmaceutical composition of claim 10, wherein the pharmaceutical composition comprises:
from 1 wt % to 15 wt % of the excipient granulation; and
from 85 wt % to 99 wt % of the pharmaceutical granulation,
wherein wt % is based on the total weight of the pharmaceutical composition.

12. The pharmaceutical composition of claim 10, wherein,
the excipient granulation has a first bulk density;
the pharmaceutical granulation has a second bulk density; and
the first bulk density is within ±20% of the second bulk density.

13. A pharmaceutical product comprising the pharmaceutical composition of claim 8.

14. The pharmaceutical product of claim 13, wherein the pharmaceutical product comprises the excipient granulation, the pharmaceutical granulation, or a combination thereof contained within a package.

15. The pharmaceutical product of claim 14, wherein the package comprises a sachet.

16. The pharmaceutical product of claim 14, wherein the pharmaceutical product comprises the excipient granulation and a pharmaceutical granulation combined in the same package.

17. The pharmaceutical product of claim 15, wherein the pharmaceutical product comprises a first package containing the excipient granulation and a second package containing the pharmaceutical granulation.

18. An oral pharmaceutical composition for oral administration prepared using the pharmaceutical composition of claim 10, wherein the oral pharmaceutical composition comprises a viscous aqueous solution having a viscosity from 50 cP to 1,000 cP, where the viscosity is determined using a Brookfield Digital Viscometer Model LVDV-I+ equipped with a Brookfield SC4-18 spindle at a spindle speed of 30 rpm at a temperature of 23° C.

19. The oral pharmaceutical composition of claim 18, wherein the oral pharmaceutical composition comprises a pharmaceutical granulation suspended in the viscous aqueous solution.

20. The oral pharmaceutical composition of claim 18, wherein the oral pharmaceutical composition comprises an active pharmaceutical ingredient suspended in the viscous aqueous solution.
